(19) United States
(12) Patent Application Publication
(10) Pub. No.: US 2018/0064821 A1
(43) Pub. Date: Mar. 8, 2018

(54) SITE SPECIFIC CURCUMIN-POLYMER MOLECULAR COMPLEXES AND METHODS OF TREATING COLON DISEASES AND INFLAMMATION

(71) Applicant: South Dakota Board of Regents, Brookings, SD (US)

(72) Inventors: Hemachand Tummala, Brookings, SD (US); Siddharth Kesharwani, Brookings, SD (US)

(21) Appl. No.: 15/695,757

(22) Filed: Sep. 5, 2017

Related U.S. Application Data
(60) Provisional application No. 62/383,124, filed on Sep. 2, 2016.

(51) Int. Cl. A61K 47/58; A61K 31/12

(52) U.S. Cl. CPC A61K 47/58; A61K 31/12

(57) ABSTRACT

Methods and materials relating to a medicament preparation comprising a curcuminoid component, such as curcumin, and a polymer component having a backbone comprising polymethacrylate or methyl methacrylate provided as a curcuminoid-polymer complex, which enhances the solubility, stability and bioavailability of the curcumin component and are useful for the treatment of various inflammatory diseases and conditions when delivered to the gastro-intestinal tract, including sepsis, mucositis, gastritis, infections, inflammatory bowel disease and cancers of GIT. The curcumin-polymer complex inhibiting the activation of TLR receptors and thereby reduce the release of inflammatory cytokines, such that the curcumin-polymer complexes are more potent than free curcumin in antagonizing on the activation of TLR4.

### CROSS-REFERENCE TO RELATED APPLICATION

The present application claims the benefit of U.S. Provisional Application No. 62/383,124, filed Sep. 2, 2016, which is hereby incorporated herein in its entirety by reference.

### TECHNICAL FIELD

The present invention relates generally to curcumin compositions and/or other curcuminoid compositions useful for the treatment of various diseases and conditions including sepsis, mucositis, enteritis, GI acute radiation syndrome, inflammatory bowel disease, irritable bowel syndrome, colon cancer, neurodegeneration, inflammation, and immunodeficiency. In particular, the present invention relates to the material and methods involving curcuminoid formulations, such as curcumin, having a complex formed with a polymethacrylate or methyl methacrylate-based polymer, which has enhanced stability, aqueous solubility and/or bioavailability. The curcumin-polymer molecular complex inhibiting the stimulation by TLR stimulants such as Monophosphoryl Lipid A (MPLA) or bacteria (E. Coli).

### BACKGROUND

For many therapeutic compounds to achieve effective bioavailability and solubility, they must dissolve in gastric fluid and permeate intestinal membranes. The efficacy of therapeutic compounds is generally hindered if they are metabolized too rapidly before, during, or immediately after absorption. For example, the generally low bioavailability of curcumin is attributed to its extremely low aqueous solubility and high rate of metabolism. However, once solublized, curcumin is capable of being effectively absorbed through intestinal membranes and has great potential to prevent and treat a wide spectrum of diseases such as cancer, Alzheimer's disease, inflammatory bowel syndrome, arthritis, etc. Regular consumption of curcumin or curcuminoids has been shown to delay or prevent these diseases. For example, current research has shown that the anti-cancer properties of curcumin may be due to its inhibition of NFKB activation, JNK and AP-1 transcriptional activity. It is well documented that curcumin acts as a potent inhibitor of NFKB signaling pathway which is involved in apoptosis as well as its function has been implicated in inflammation, cell proliferation, differentiation and cell survival. Although curcumin possesses anti-cancer and anti-inflammatory properties, among others, it is still considered extremely safe when administered at very high doses. Conversely, systemic toxicity at high dose rendered other anti-cancer drugs unsuitable for cancer therapy. It was recently reported that uptake of curcumin is safe at doses ranging from 3600-8000 mg/day for four months. In one clinical trial, no toxic effects were seen in patients taking curcumin at a dose of 8 g/day for 18 months.

Inflammatory bowel disease (IBD) is an umbrella term comprising of Crohn's disease and ulcerative colitis is a chronic lifelong condition severely affecting the quality of life. It is an inflammatory condition causing inflammation, mucosal ulceration, edema and the hemorrhage of gastrointestinal (GI) tract and colon. These conditions can only be treated but cannot be cured. The etiology behind IBD is not clearly understood, however it is hypothesized to be caused due to genetic predisposition, immune system disturbance and environmental factors. The current treatment strategy for IBD employs anti-inflammatory drugs, antibiotics, biologics and immunosuppressive agents. However serious adverse effects, cost and need for the systemic delivery demonstrate the unmet necessity for treatment modalities with high local efficacy and low systemic toxicity. The current IBD treatment/therapy should aim to be directed towards specific targeting to the inflamed colon tissue via oral administration to increase the therapeutic efficacy by providing local concentrations of the drug and keeping the inflamed tissue in contact with the treatment for longer duration of time thereby decreasing the systemic adverse effects.

Despite recent advances in medicine, drug delivery and nanotechnology, colorectal cancer is the third most common cancer diagnosed in the United States. American cancer society estimates ˜95,000 new colo-rectal cancer cases in 2016. Therefore the need to develop novel alternative therapeutic and preventive strategies remains an important goal.

Colon targeted delivery systems have been in focus for numerous years due to its potential to improve treatment of local diseases affecting the colon while minimizing the systemic side effects. Various approaches have been investigated for colonic delivery including prodrugs, colon targeting by coatings, biodegradable and bioadhesive delivery systems, matrix based systems, time release systems, multiparticulate delivery systems, and polysaccharide based systems. Most of the studies reported in the literature for colon specific delivery are based on one of the following approaches (i) prodrug based approaches (ii) pH-dependent systems (iii) microflora assisted systems and (iv) time-dependent systems.

Conventional oral formulations which have been designed to achieve systemic delivery of therapeutics suffer from adverse effects and toxicity due to distribution in other organs of the body. One of the approaches that could be exploited to develop more efficacious and safer therapeutic modalities include developing targeted drug delivery system to achieve high amounts of drug concentration locally at the site of action thus having minimal to zero exposure to healthy or distant tissues. Providing higher local concentrations of the drug to the site will minimize frequent drug dosing thus achieving higher patient compliance. Thus, oral formulation that achieve localized drug concentrations are preferred in the design of colon targeted delivery systems. Therefore, exploiting the physiological conditions of the GIT, in particular the colon, should be one of the aims in designing delivery systems. Locally acting delivery systems administered orally should protect the drugs from gastric degradation, intestinal metabolism and enzymatic degradation at the lower part of the gastro intestinal tract (GIT) thus providing improved targeting at the colon tissue.

Numerous nanoparticulate studies of chitosan, alginate, Eudragit® S100 or Eudragit® RS polymer has been reported with curcumin, ciprofloxacin or other drugs for colon targeting. Previously described formulation studies focused on cyclodextrin complexes, microspheres, microsponges, solid-lipid nanoparticles, inclusion complexes of curcumin etc. These studies however have failed to overcome the solubility and stability issues of curcumin to deliver local, soluble and biologivcally potent curcumin to the desired location in the digestive tract to advance the therapeutic potential of curcumin for diseases such as cancer, Alzheimer's disease, inflammatory bowel syndrome, arthritis, sepsis and colo-rectal cancer.

Curcumin is a natural dietary polyphenolic compound extracted from the rhizomes of turmeric Curcumin longa. It has demonstrated evidence in various types of complex chronic diseases such as cancer, inflammatory bowel diseases, atherosclerosis, diabetes, malaria, alzheimer's disease and arthritis. These are chronic diseases that need interference in multiple pathways due to their complex nature. The unique advantage of curcumin lies in the multiple pharmacological activities it possess that interfere at multiple pathways in the above mentioned chronic diseases. Importantly, curcumin is generally recognized as safe (GRAS) by the United States Food and Drug Administration (US-FDA) for daily use. Despite it demonstrated efficacy and safety in animal models of various diseases, the clinical advancement of curcumin as a local and oral therapy is hindered due to its extremely poor water solubility (<1 μg/ml), degradation in aqueous fluids, rapid metabolism in the body and short biological half-life after oral administration. Thus, because of these limitations, it becomes difficult to maintain appropriate amounts of local and soluble curcumin levels at the site of action for longer duration. Hence solubility becomes an important factor in the delivery of curcumin and numerous scientific studies have been performed to improve the solubility, thus deliver biologically potent curcumin to the site of action. These strategies include but not limited to structural modification, chelation and bioconjugates, use of adjuvants which block the curcumin metabolism, formulating into complex dosage forms such as, micelles, liposomes, polymeric nanoparticles, lipid nanoparticles, cyclodextrin complexes etc. Although some of these strategies showed improvements they have not been translated into daily human use. This is due to high cost of the materials/formulations, toxicity associated with the formulations, and/or incompatibility for oral use. Therefore, there is an unmet critical need for a formulation that enhances the solubility and deliver biologically potent curcumin in a safe, economical, and practical for daily oral use.

Despite its therapeutic benefits and non-toxicity at high doses, curcumin has restrictive clinical application because of its extremely low aqueous solubility, rapid systemic metabolism and degradation at alkaline pH, which severely curtails its bioavailability and functionality. With respect to solubility, curcumin shows extremely low solubility in aqueous solutions (less than 1 μg/ml in water without any solubility enhancement techniques), but it is soluble in organic solvents such as DMSO, ethanol, methanol, and acetones. Its degradation kinetics have also been reported under various pH conditions, showing relative stability at acidic pHs (i.e., stomach) but unstable at neutral and basic pHs. It has also been reported that most curcumin (>90%) is rapidly degraded within 30 min at pH 7.2 and above. Studies have suggested that this low aqueous solubility, high degradation of curcumin at physiological pHs, and faster metabolism consequently leads to poor absorption, low tissue distribution, and rapid excretion of curcumin that severely restrict its bioavailability. Therefore, a patient must consume large doses of curcumin and curcuminoids in order to achieve detectable serum concentrations needed for its therapeutic benefits. Additionally, the low bioavailability and solubility of curcumin hinders the incorporation of curcumin and curcuminoids into effective pharmaceutical and nutraceutical formulations for both animals and humans. In addition, it is important for the drug to be in a soluble form for biological activity. Therefore, insoluble curcumin is biologically inactive even at the luminal side of GIT.

To address the issues of low aqueous solubility and bioavailability of curcumin, several approaches have been explored, including the use of adjuvants to delay its metabolism and the use of excipients to enhance its bioavailability. However, the adjuvants and excipients thus far identified are not compatible with the use of curcumin and curcuminoids as a regular supplement due to their high costs and lack of practicability. Although adjuvants have been shown to delay or inhibit the metabolism of curcumin and curcuminoids, inhibiting or delaying their metabolism alone without enhancing their solubility will not result in an effective formulation. Therefore, there is a need for a formulation that enhances the bioavailability, solubility and stability issues of curcumin, such that the therapeutic benefits of this compound can be fully realized. For local diseases of the intestinal tract, there is no need to expose the whole body with the drug. There is also a strong need to deliver local, soluble and biologically potent curcumin to the colon tissue without exposing the other organs of the body thus advancing the therapeutic potential of curcumin for diseases and conditions of GIT, including mucositis, IBD, sepsis and colo-rectal cancer.

The information included in this Background section of the specification is included for technical reference purposes only and is not to be regarded as subject matter by which the scope of the description is to be bound or as an admission of prior art.

### SUMMARY

The present invention relates generally to curcumin and/or curcuminoid formulations useful for the treatment of various diseases including cancer, neurodegeneration, inflammation, and immunodeficiency. Specifically, the present invention provides a composition comprising one or more curcuminoid-polymer complexes, the one more curcuminoid-polymer complexes presenting enhanced stability, aqueous solubility, GIT targetability and improved bioavailability of the curcuminoid component. Curcumin-Eudragit® molecular complexes (CEMCs), which when curcumin complexes with the polymer comprising Eudragit® S100 and Eudragit® EPO are referred herein as Ora-Curcumin-S and Ora-Curcumin-E, respectively.

In some aspects, the curcuminoid component is chosen from curcumin, demethoxycurcumin, bisdemethoxycurcumin, tetrahydroxycurcumin, Bis-0-Demethyl curcumin (BDMC), other derivatives of curcuminoids, or combinations thereof.

In some aspects, the polymer component comprises a polymer or co-polymer having a backbone comprising polymethacrylate or methyl methacrylate, such that the polymer component may have various different functional side-chains attached thereto.

In some aspects of the present invention, a medicament preparation comprises a curcuminoid component and a polymer component, the curcuminoid component chosen from curcumin, demethoxycurcumin, bisdemethoxycurcumin, tetrahydroxycurcumin, Bis-0-Demethyl curcumin (BDMC), derivatives of curcuminoids, or combinations thereof, wherein at least a portion of the curcuminoid component and at least a portion of the polymer component are in the form of a curcuminoid-polymer complex.

In some aspects of the present invention, the polymer has a backbone of a polymethacrylate-based copolymer or methyl methacrylate-based copolymer. In some aspects, the polymethacrylate-based copolymer is anionic, cationic or a neutral copolymer.

In some aspects of the present invention, the medicament preparation further comprises an adjuvant. In some aspects, the adjuvant is a P-glycoprotein inhibitor or an inhibitor of glucouronidation. In some aspects, the adjuvant is piperine.

In some aspects of the present invention, the curcuminoid component of the medicament preparation has a solubility in water in an amount greater than 1 μg/ml, in some aspects greater than 5 μg/ml, in some aspects greater than 10 μg/ml, in some aspects greater than 20 μg/ml, in some aspects greater than 30 μg/ml, in some aspects greater than 40 μg/ml, in some aspects greater than 50 μg/ml, in some aspects greater than 75 μg/ml, in some aspects greater than 100 μg/ml, in some aspects greater than 200 μg/ml, in some aspects greater than 300 μg/ml, in some aspects greater than 400 μg/ml, in some aspects greater than 500 μg/ml, in some aspects greater than 600 μg/ml, in some aspects greater than 700 μg/ml, in some aspects greater than 800 μg/ml, in some aspects greater than 900 μg/ml, in some aspects greater than 1 mg/ml, in some aspects greater than 5 mg/ml, in some aspects greater than 10 mg/ml, in some aspects greater than 15 mg/ml, in some aspects greater than 20 mg/ml, and in some aspects greater than 50 mg/ml.

In some aspects, the aqueous solubility of the curcuminoid component of the medicament preparation is between about 1 μg/ml and about 50 mg/ml, in some aspects between about 10 μg/ml and about 40 mg/ml, in some aspects between about 100 μg/ml and about 20 mg/ml, and in some aspects between about 1 mg/ml and about 20 mg/ml.

In some aspects, the aqueous solubility of the curcuminoid component of the medicament preparation is between about 1 mg/ml and about 100 mg/ml, in some aspects between about 10 mg/ml and about 25 mg/ml, in some aspects between about 10 μg/ml and about 50 mg/ml, and in some aspects between about 25 mg/ml and about 50 mg/ml.

In some aspects of the present invention, the medicament preparation can further comprise an amphipathic component or surfactant or polymer that facilitates the formation of curcumin-polymer complexes that can nanoparticles or microparticles. In some aspects, the surfactant/stabilizer is (e.g., polyvinyl alcohol or PVA), which can enhance the loading of curcumin as well as aid in the solubilization process.

In other aspects, the medicament preparation further comprises other adjuvants, excipients, nutraceuticals, and/or pharmaceuticals providing additional therapeutic benefits.

In some aspects of the present invention, the medicament preparation is in the form of a solid (e.g., tablet or pill), a liquid (e.g., solution, suspension or lotion), or semisolid (e.g., gel, cream or ointment). In other embodiments, the composition of curcumin-polymer complexes can be injected into the patient's body. In still other embodiments, the composition of curcumin-eudragit complexes can be used orally, injected through parenteral route, applied topically to the patient's skin, or inserted into a patient's bodily orifice such as intranasally, through pulmonary administration, through ophthalmic delivery, delivering rectally or vaginally.

In some aspects of the present invention, a curcumin formulation with enhanced bioavailability comprises a complex comprising a curcumin and/or a curcuminoid component and a polymer or copolymer component, the polymer or copolymer compoing having a backbone comprising polymethacrylate or methyl methacrylate. In some aspects, the polymer or copolymer is a polymethacrylate-based copolymer, and further includes anionic, cationic, and neutral copolymers (e.g., Eudragit® EPO or Eudragit® S-100) that enhances the solubilization of curcumin. In other aspects, the curcumin used in the formulation can exist as -keto or -enol forms, or the curcumin used can be a combination of different commercially available forms of curcumin. In some aspects, the curcuminoid comprises curcumin, demethoxycurcumin, bisdemethoxycurcumin, tetrahydroxycurcumin, Bis-0-Demethyl curcumin (BDMC), or combinations thereof. In some aspects, the polymer or compolymer and the curcumin used in the formulation can form a complex based on intermolecular interactions (e.g., hydrophobic interactions or hydrogen bonding), which enhances the aqueous solubility, stability and/or the bioavailability of the curcumin and/or curcuminoid component. In some aspects, the polymer or co-polymer and the curcuminoid used in the formulation can form a complex based on intermolecular interactions (e.g., hydrophobic interactions or hydrogen bonding or polar interactions), which enhances the aqueous solubility, stability and/or the bioavailability of the curcumin and/or curcuminoid component or provides tissue specific targeting ability to curcumin and/or curcuminoid components.

In some aspects of the present invention, the formulation further comprises an adjuvant that delays or inhibits curcumin metabolism. In some aspects, the adjuvant is a P-glycoprotein inhibitor or an inhibitor of glucouronidation. In some aspects, the adjuvant can be piperine. Delaying the metabolism of curcumin using piperine enhances its therapeutic effects by enabling it to persist longer in the patient and provides more time for penetration into target tissues.

In some aspects, the present invention features a method of forming a medicament preparation comprising particulate formulations (i. e., nanoparticles/microparticles) loaded with curcumin-polymer complexes having enhanced stability, aqueous solubility and/or bioavailability using sonication and precipitation techniques.

In some aspects, curcumin and/or other curcuminoids and a polymer can be dissolved and then added to an aqueous solution containing a surfactant. In some cases, this process can be performed using sonication or precipitation techniques. The resulting nanoparticles or microparticles can be collected and analyzed for the amount of curcumin accumulation present in the particles. In some cases, different formulation and process parameters (e.g., type and concentration of organic solvent or surfactant used, curcumin to polymer ratio, etc.) can be used in order to alter the formulation to obtain increased or decreased loading and/or increased or decreased solubility. For example, in some cases, the particle size can be altered by changing the surfactant type (e.g., Tween-20, Pluronic F68, or polyvinyl alcohol) and the surfactant concentration (1%, 2%, or 3% w/v) in the preparation. The amount of curcumin and/or other curcuminoid accumulation present in the particles can be enhanced by changing the ratio between the curcumin and/or other curcuminoid with the polymer (e.g., 1:5, 1:3, or 1:2) to which it forms a complex. Other parameters can also be altered to change the size of the particles, including the amount of energy used during sonication and total sonication time.

In some aspects, the curcuminoid component and the polymer component can form a curcuminoid-polymer complex by melting the curcuminoid component and the polymer component together as a hot melt and cooling together (e.g., hot-melt extrusion).

In some aspects of the present invention, a method of treating patients for diseases involving cancer, neurodegeneration, inflammation, and immunodeficiency comprises administering a medicament preparation comprising nanoparticles loaded with curcumin-polymer complexes, the medicament preparation having enhanced bioavailability of the curcumin component. The curcumin-polymer complexes of the present invention can enhance the bioavailability of curcumin up to or more than 20,000 times compared to free curcumin.

In some aspects of the present invention, a method of treating patients for diseases involving the activation of Toll-like receptors (TLRs) or mediated by the inflammatory cytokines such as TNF-alpha.

In some aspects of the present invention, a method of treating patients for intestinal diseases including mucositis, bacterial infections, gastritis, inflammatory bowel disease, Celiac disease, irritable bowel syndrome and/or cancers of GIT comprises administering a medicament preparation to a subject, the medicament preparation comprising a curcumin-polymer complex.

In some aspects of the present invention, a method of using the curcumin-polymer complexes for the improvement of general intestinal health.

In some aspects of the present invention, the curcumin-polymer complexes are used alone, with other adjuvants or as a component with various food products such as soups, beverages, puddings, lollipops, candies, chewing gums and the like.

In some aspects of the present invention, a method of inhibiting the stimulation of TLR receptors in a patient includes providing a curcuminoid-polymer complex in a medicament, the curcuminoid-polymer complex comprising at least one curcuminoid component selected from the group consisting essentially of curcumin, demethoxycurcumin, bisdemethoxycurcumin, tetrahydroxycurcumin, Bis-0-Demethyl curcumin (BDMC), or combinations thereof, at least one polymer or co-polymer component having a backbone comprising polymethacrylate or methyl methacrylate, and optionally a surfactant component, and delivering the curcuminoid-polymer complex to a gastro-intestinal tract of the patient.

In some aspects, the the curcuminoid-polymer complex is delivered to the gastro-intestinal tract of the patient by administering a dosage from about 0.1 mg/kg/day to about 1 mg/kg/day, in some aspects administered in a dosage of about 200 mg/day to about 15,000 mg/day. The dosage to be administered can comprise, for example, curcumin in an amount of from about 1.05 to about 85 mg/kg patient body weight, or from about 8.8 to about 13.4 mg/kg body weight, or from about 11.1 to about 111 mg/kg patient body weight, or from about 88.8 to about 133.2 mg/kg patient body weight.

In some aspects, the method comprises administering a first curcuminoid-polymer complex and a second curcuminoid-polymer complex, wherein the first curcuminoid-polymer complex has a first polymer or co-polymer component and the second curcuminoid-polymer complex has a second polymer or co-polymer component, the first polymer or co-polymer forming a first complex with a curcuminoid component and the second polymer or co-polymer forming a second complex with a curcuminoid component, wherein the first polymer or co-polymer component is chosen such that the aqueous solubility of the curcuminoid component of the first complex is greater than about 1 μg/ml at a pH between about 1.0 and about 5.0, and wherein the second polymer or co-polymer component is chosen such that the aqueous solubility of the curcuminoid component of the second complex is greater than about 1 μg/ml at a pH between about 5.5 and about 14.0.

In some aspects, the weight ratio of the curcuminoid component to the polymer or co-polymer component used in a preparation of the curcuminoid-polymer complex is between about 1:0.1 to about 1:50.

In some aspects, the curcuminoid-polymer complex increases the stability of the curcuminoid component in an aqueous solution compared to free curcumin.

In some aspects, the curcuminoid component of the curcuminoid-polymer complex is curcumin, which when delivered to the gastro-intestinal tract inhibits the activation of TLR receptors. In some aspects, the curcumin of the curcuminoid-polymer complex delivered to the gastro-intestinal tract antagonizes TLR4 activation. In some aspects, the curcumin of the curcuminoid-polymer complex delivered to the gastro-intestinal tract reduces the release of pro-inflammatory cytokines by immune cells.

In some aspects, a method of treating a disease or disorder mediated by inflammatory cytokines in a patient includes administering to a patient a medicament having a plurality of curcuminoid-polymer complex particles, the curcuminoid-polymer complex comprising at least one curcuminoid component selected from the group consisting essentially of curcumin, demethoxycurcumin, bisdemethoxycurcumin, tetrahydroxycurcumin, Bis-0-Demethyl curcumin (BDMC), or combinations thereof, at least one polymer or co-polymer component having a backbone comprising polymethacrylate or methyl methacrylate, and a surfactant component, wherein an aqueous solubility of the curcuminoid component of the curcuminoid-polymer complex is greater than about 1 μg/ml at a pH between about 2.0 and about 14.0.

In some aspects, the disease or disorder that is treated by is selected from mucositis, sepsis, gastritis, irritable bowel syndrome, inflammatory bowel disease, bacterial infections, and cancers of the gastro-intestinal tract. In some aspects, the curcuminoid-polymer complex is ingested such that it enters the gastro-intestinal tract of the patient.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the present invention will be apparent from the following detailed description, and from the claims.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is an illustration of the molecular complex formed between curcumin and a Eudragit™ polymer in order to increase the stability, aqueous solubility and/or bioavailability of curcumin, according to certain aspects of the present invention. This illustration is also representative of other curcuminoids alone or in combination of curcumin that can form a complex with a Eudragit™ polymer according to certain other aspects of the present invention.

FIG. 2A presents a graph relating to a sample of (i) free curcumin dissolved in ethanol, and (ii) samples of the complex formed between curcumin and a polymer (Eudragit® EPO) dissolved in an aqueous solution at pH 1.2 and 4.5, respectively, according to certain aspects of the present invention. The free curcumin and curcumin-polymer (Eudragit® EPO) drug complex (“Ora-Curumin-E”) having been passed through a 10 KDa molecular weight cut-off membrane, such that curcumin that is smaller and polymers that are higher than 10 KDa in their molecular weight, resulted in free curcumin passing freely through the membrane unless it formed a complex with the high molecular weight polymer, such as in the curcumin-polymer complex illustrated in FIG. 1. The ratio of the concentration of dissolved curcumin before and after filtration was determined by UV-spectrophotometry and plotted on the Y-axis. As shown in the present graph, a ratio of about 1 indicates no complex formation, such as in the free curcumin dissolved in ethanol, while a ratio less than 1 indicates complex formation between curcumin and the polymer (Eudragit® EPO).

FIG. 2B presents a graph and illustrative assessment of curcumin-polymer (Eudragit® 5100) drug complex (“Ora-Curcumin-S”) formation, wherein curcumin and curcumin equivalent of Ora-Curcumin-S were dissolved in absolute ethanol, 10% and 0% v/v ethanol in 50 mM phosphate buffer pH 7.0 as shown in the figure. The ratio of the concentration of soluble curcumin present in the solution after passing through the 10 kDa cut-off membrane (bottom) to the concentration measured before filtration (top) was compared. Data represent mean±standard deviation (n=3-4). Complex formation with high molecular weight Eudragit® S100 (˜125 kDa) will prevent curcumin with a molecular weight of 0.368 KDa to filter through the membrane with 10 kDa cutoff limit.

FIG. 3 presents a set of graphs relating to the effects of organic solvents on the formation of the curcumin-polymer complexes, according to certain aspects of the present invention, with the polymer comprising Eudragit® EPO, and the data represent mean+standard deviation. Loading represents μg of curcumin present in 1 mg of the formulation, and the “*” indicates statistical significance (P<0.05) as compared to the ethyl acetate group.

FIG. 4 presents a set of graphs relating to effects of surfactants on the formation of the curcumin-polymer complexes, according to certain aspects of the present invention, with the polymer comprising Eudragit® EPO, and the data represent mean+standard deviation. Loading represents μg of curcumin present in 1 mg of the formulation, and the “*” indicates statistical significance (P<0.05) as compared to pluronuc F-68.

FIG. 5 presents a graph relating to the effect of increasing amounts of curcumin (mg) to polymer ratio on the loading of curcumin in the final curcumin-polymer complexes, according to certain aspects of the present invention, with the polymer comprising Eudragit® EPO, and the data represent mean+standard deviation. Loading represents μg of curcumin present in 1 mg of the formulation, with up to 50 mg of curcumin being capable of being added per 100 mg of the Eudragit™ polymer, according to certain aspects of the present invention.

FIG. 6A presents a graph with corresponding images of aqueous solubility of curcumin measured in mg/ml at pH 1.2, 4.5, and 7.4, according to certain aspects of the present invention, with a solution with a yellow color indicating fully dissolved curcumin (i.e., left panel, first and second solutions), while a clear solution indicating absence of soluble curcumin (i.e., left panel, third solution; right panel, first, second, and third solutions). The concentration of dissolved curcumin in water in the shown bottles being represented in the corresponding bar graph below. The left panel represents curcumin-polymer (Eudragit-EPO) complexes and the right panel represents free uncomplexed curcumin.

FIG. 6B illustrates that aqueous soluble cucumin-polymer complexes can be made with other polymers other than Eudragit® EPO. Curcumin complexes were made with both Eudragit® EPO and Eudragit® S100 as the polymer component. The aqueous solubility of these complexes depends on the chemical groups on the side chain of the polymer and the pH of the aqueous solution. Images of curcumin solubility measured in mg/ml at pH 1.2, 4.5, 7.0, and 7.4, according to certain aspects of the present invention, with a solution with a yellow color indicates fully dissolved curcumin (i.e., top panel of curcumin-polymer (Eudragit® 5100) complexes, third and fourth solutions; middle panel of curcumin-polymer (Eudragit® EPO) complexes, first and second solutions), while a clear solution indicates absence of soluble curcumin (i.e., top panel of curcumin-polymer (Eudragit® 5100) complexes, first and second solutions; middle panel curcumin-polymer (Eudragit® EPO) complexes, third and fourth solutions; bottom panel of free curcumin, first, second, third, and fourth solutions).

FIG. 7 presents a graph of the kinetic solubility of curcumin in the form of (i) free curcumin, (ii) a physical mixture of blank polymer (Eudragit® EPO nanoparticles—only the polymer and surfactant) and free curcumin, and (iii) nanoparticles loaded with curcumin-polymer (Eudragit® EPO) complexes, according to certain aspects of the present invention, which were dispersed in 1 ml pH 1.2 buffer and incubated at 37 C for 4 hrs with 100 rpm shaking, the curcumin dissolved after 4 hours being spectrophotmetrically analyzed from different samples, with the data representing mean+standard deviation (n=3), and the “*” indicating results that are statistically significant (P<0.05) using a Student t-test.

FIG. 8 presents a graph of the stability of aqueously soluble curcumin-polymer complexes at pH 1.2, 4.5, 6.8, or 7.4, according to certain aspects of the present invention, with free curcumin or the equivalent amount of curcumin-polymer (Eudragit® EPO) complexes solubilized in 10% methanol in each respective buffer and incubated at 37 C. At different time points, the samples were centrifuged (20,000 g), and the supernatant was passed through 0.2 μm filter, and the amount of soluble and stable curcumin in the filtrate was determined by UV absorbance at 420 nm. The data indicates that curcumin-polymer complexation enhanced the stability of curcumin in aqueous buffers.

FIG. 9 presents a graph measuring the oral bioavailability of curcumin in mice, according to certain aspects of the present invention, with free curcumin (150 mg/kg) or the equivalent amount of curcumin-polymer (Eudragit® EPO) complexes being orally administered to mice, and blood samples being collected from mice at different time points, and curcumin extracted from the plasma and analyzed via by HPLC with UV absorption at 420 nm.

FIG. 10A is a schematic representation of in vitro skin penetration studies performed and referenced in FIG. 10B, according to certain aspects of the present invention, with free curcumin (10 mg/ml) or the equivalent amount of curcumin-polymer (Eudragit® EPO) complexes being added to the donor chamber in about pH 4.5 buffer, and the amount of curcumin transported across the porcine ear skin (represented as membrane in the schematic) being determined by measuring the amount of curcumin in the acceptor compartment by HPLC with UV absorption at 420 nm.

FIG. 10B presents a graph measuring the topical bioavailability of curcumin, according to certain aspects of the present invention, with the in-vitro skin penetration studies being performed using porcine skin, and free curcumin (10 mg/ml) or the equivalent amount of curcumin-polymer (Eudragit® EPO) complexes being added to the donor chamber in pH 4.5 buffer, the amount of curcumin transported across the skin being determined by HPLC, the amount of curcumin that permeated the skin measured in μg/cm2, and the data representing cumulative amounts, with the final time point being 24 hours after the experiment having been initiated.

FIG. 11 illustrates that the pH dependent aqueous solubility of curcumin-polymer molecular complexes (Ora-Curcumin-S or Ora-Curcumin-E), according to certain aspects of the present invention, and of free curcumin. The images show curcumin solubility measured in mg/ml at pH 1.2, 4.5, 7.0, and 7.4. A solution with a yellow color indicates fully dissolved curcumin (i.e., third and fourth solutions in the top panel, first and second solutions of the middle panel), while a clear solution indicates absence of significant amounts of soluble curcumin (in rest of the solutions). Ora-Curcumin-S and Ora-Curcumin-E represent molecular complexes of curcumin with Eudragit® S100 and Eudragit® EPO polymers, respectively.

FIG. 12 presents a Fourier transform infrared spectroscopy (FTIR) spectrum of curcumin (in blue (second from top)), Eudragit® S100 polymer (in red (top)), physical mixture of curcumin and Eudragit® S100 (in cyan (third from top)) and curcumin-polymer complexes (CEMCs) (in green (bottom)), according to certain aspects of the present invention. FTIR spectrum of curcumin, Eudragit® S100 polymer, the physical mixture of curcumin and Eudragit® S100, and curcumin-polymer (Eudragit® S100) molecular complexes were performed using Nicolet 380 ATR-FTIR spectrophotometer (Thermo Electron Corp., Madison, Wis.). The data was acquired between 4000 cm-1 and 400 cm-1 at a scanning speed of 4 cm-1 and 50 scans. The average of 50 scans data was presented. As can be seen in CEMCs (in green (bottom)), the hydroxy (—OH) band of curcumin was diminished in CEMCs. The data from this figure indicates that a possible involvement of hydrogen bonding in the curcumin-polymer complex formation.

FIG. 13 presents a proton nuclear magnetic resonance (¹HNMR) spectrum of curcumin-polymer (Eudragit® S100) molecular complex, according to certain aspects of the present invention. ¹HNMR spectrum of Ora-Curcumin-S was recorded using Bruker 400 MHz NMR spectrometer. Ora-Curcumin-S (30 mg) was taken in 1 ml of D₂O: 0.5N Na₂CO₃ (1:1), complex dissolved in basic D₂O (by vortexing) and ultra-centrifuged to gives a clear supernatant. This supernatant was used for NMR. A)¹H NMR of the supernatant. B)¹H NMR of 400 μl of the supernatant followed by dilution with 400 μl of DMSO-d₆. The data from this figure indicates that hydrophobic interactions are involved in the curcumin-polymer complex formation, which were disturbed in DMSO.

FIG. 14A presents a scanning electron micrograph of CEMCs, according to certain aspects of the present invention, taken at 500× magnification, 5 kV accelerating voltage, and where the dry samples were mounted on metal holders using conductive double-sided tape and sputter coated with a gold layer for analysis.

FIG. 14B presents a pictograph of lyophilized CEMCs, according to certain aspects of the present invention.

FIG. 15 presents differential scanning calorimetry (DSC) curves of (A) Curcumin, (B) Eudragit® S100 polymer, (C) Physical mixture of curcumin and Eudragit® S100 polymer, and (D) curcumin-polymer complexes (CEMCs) (Ora-Curcumin-S), according to certain aspects of the present invention. The X-axis represents temperature in ° C. whereas the Y-axis represents the heat flow in W/g. Samples were weighed (equivalent to curcumin) and placed in sealed Tzero aluminum hermetic pans. With liquid nitrogen as coolant, samples were scanned at 10° C./min from −20° C. to 300° C. and thermograms were recorded.

FIG. 16 presents X-ray diffraction patterns (XRD) curves of (A) Curcumin, (B) Eudragit® S100 polymer, (C) Physical mixture of curcumin and Eudragit® S100 polymer, and (D) curcumin-polymer complexes (CEMCs) (Ora-Curcumin-S), according to certain aspects of the present invention. The X-axis represents 2θ degree and the Y-axis represents intensity in cps. Samples were mounted on double sided silicone tape and measurements were performed from 2° to 60° at a scan speed of 4°/min and increments of 0.02°.

FIG. 17 illustrates the aqueous stability of Ora-Curcumin-S. The aqueous stability of curcumin or curcumin equivalent of Ora-Curcumin-S was determined in pH 7.0 buffer and simulated intestinal fluid (SIF, pH 6.7-6.9) for a period of 24 h. Data represents mean±standard deviation (n=3-4). The data indicates that curcumin complexation with polymers enhanced the stability of curcumin in aqueous buffers.

FIG. 18 presents a graph measuring the inhibitory effect of curcumin-polymer complexes (CEMCs) on Toll-Like receptor-4 (TLR-4) activity tested on HEK293-TLR4^{YFP}-MD2 cells according to certain aspects of the present invention, as shown by reduced IL-8 release. Data represents mean±standard deviation (n=3). The X-axis represents the drug concentration in μg/mL and the Y-axis represents the IL8 release in pg/mL. A total of 5.0×10⁵ HEK293-TLR4^{YFP}-MD2 cells were seeded in a 24-well plate using DMEM-high glucose supplemented with 10% fetal bovine serum (FBS), penicillin (100 U/ml) and streptomycin (100 μg/ml). The cells were treated with four different groups a) Medium only b) MPLA (2 μg/ml) c) curcumin (2-50 μg/ml) d) curcumin equivalent of curcumin-polymer (Eudragit® S100) complexes, CEMCs (2-50 μg/ml). Post 1 h of incubation, the cells were treated with MPLA (2 μg/ml). After 48 h of treatment, the concentration of IL-8 was measured in the culture supernatant using Human IL-8 ELISA Ready-SET-Go kit (e-bioscience, San Diego, Calif.). MPLA is a known TLR-4 agonist that specifically activates TLR-4 receptors on HEK293-TLR4^{YFP}-MD2 to release IL-8 in the medium. CEMCs are significantly better than soluble curcumin in inhibiting MPLA-induced TLR-4 signaling in HEK293-TLR4^{YFP}-MD2 cells.

FIG. 19 presents represents an increased resistance of HEK293TLR4^{YFP}-MD2 cells against MPLA-induced activation of TLR-4 signaling in the presence of curcumin or curcumin equivalent of Ora-Curcumin-S(CEMCs) at fixed 5 μg/ml concentration, according to certain aspects of the present invention. The data represents mean±standard deviation (n=3). *** indicates that the values are significantly higher (p≦0.001) calculated using one way ANOVA, followed by Bonferroni's post-hoc multiple comparison test., The X-axis shows MPLA concentration in μg/mL and the Y-axis shows IL8 concentration in pg/mL. A total of 5.0×10⁵ HEK293TLR4^{YFP}-MD2 cells were initially treated with curcumin (5 μg/ml) or curcumin equivalent (5 μg/ml) of curcumin-polymer (Eudragit® S100) molecular complexes for 1 h, followed by the addition of varying concentrations of MPLA (3-6 μg/ml). After 48 h, the concentration of IL-8 in the culture supernatant was measured using human IL-8 ELISA Ready-SET-Go kit (e-bioscience, San Diego, Calif.).

FIG. 20 presents a flowchart showing the influence of reducing the inflammation by curcumin or curcumin equivalent of curcumin-polymer (Eudragit® S100) molecular complexes on preventing IBD, colo-rectal cancer or autoimmune diseases, according to certain aspects of the present invention.

FIG. 21 presents a graph measuring the effect of curcumin and CEMCs on TLR-4 activated (MPLA) inflammatory response (TNF-α release) in dendritic cells (DC2.4 cells), according to certain aspects of the present invention. The data represents mean±standard deviation (n=3), where the X-axis shows concentration measured in μg/mL and the Y-axis shows TNF-α release measured in pg/mL. A total of 5.0×10⁵ DC2.4 cells were incubated with curcumin or curcumin equivalent of CEMCs at 5 and 10 μg/ml concentrations in presence of MPLA (2 μg/ml). After 48 h, the levels of TNF-α in the cell supernatants was estimated by ELISA as marker for inflammatory response. The data represent mean±standard deviation (n=3). *** indicates that the values are significantly higher compared to all other groups (p≦0.001) calculated using one way ANOVA, followed by Bonferroni's post-hoc multiple comparison tests.

FIG. 22 presents a graph measuring the effect of curcumin and CEMCs, according to certain aspects of the present invention, on E. Coli induced inflammatory response (TNF-α release) in dendritic cells (DC2.4 cells). The data represents mean±standard deviation (n=3), where the X-axis shows concentration measured in μg/mL and the Y-axis shows TNF-α release measured in pg/mL. A total of 5.0×10⁵ DC2.4 cells were incubated with curcumin or curcumin equivalent of CEMCs at 5 and 10 μg/ml concentrations in presence of dead E. Coli (5×10⁴). After 48 h, the levels of TNF-α in the cell supernatants as a pro-inflammatory marker was estimated by ELISA. The data represent mean±standard deviation (n=3). *** indicates that the values are significantly higher compared to all other groups (p≦0.001) calculated using one way ANOVA, followed by Bonferroni's post-hoc multiple comparison tests.

FIG. 23 illustrates plasma levels of curcumin after 1 hour of administering (i) PBS, (ii) curcumin, (iii) curcumin formulation as Ora-Curcumin-S, and (iv) curcumin formulation as Ora-Curcumin-E orally in mice. Curcumin (15 mg/kg) or curcumin equivalent of Ora-Curcumin-S and Ora-Curcumin-E were given to mice orally. Blood samples were collected from the mice after 1 h of administration. Curcumin was extracted from the plasma and analyzed via HPLC with UV absorption at 420 nm. The data in the figure represents the plasma concentration of curcumin at 1 h. Data represents mean±standard deviation (n=3). The data indicates that Ora-Curcumin-E enhanced the absorption of curcumin into systemic circulation where as Ora-Curcumin-S remained in the lumen of the intestine to treat local conditions.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

Embodiments of the present invention are discussed in detail below. In describing embodiments, specific terminology is employed for the sake of clarity. However, the present invention is not intended to be limited to the specific terminology so selected. A person skilled in the relevant art will recognize that other equivalent parts can be employed and other methods developed without parting from the spirit and scope of the present invention. All references cited herein are incorporated by reference as if each had been individually incorporated.

“Treat” refers to preventing, curing, reversing, attenuating, alleviating, minimizing, suppressing or halting at least one of the symptoms or deleterious effects of the diseases, disorders or conditions described herein. Treatment encompasses both therapeutic treatment and prophylactic or preventative measures. Those in need of treatment include those already with the disorder as well as those in which the disorder is to be prevented. Hence, the patient to be treated may have been diagnosed as having the disorder or may be predisposed or susceptible to the disorder.

“Effective” or “therapeutically effective” means sufficient to cause at least one of a patient's symptoms to decrease in frequency and/or intensity. The symptoms that are decreased in frequency and/or intensity can include, for example, one or more adverse cognitive or physiological symptoms.

“Administer” means to deliver one or more doses of one of the compositions to a patient. The methods of the present inventions can involve administration of the composition by any means and via any route of administration that is consistent with effective treatment of one or more of the diseases described herein. For example, the methods can involve administering the compositions orally, topically on the skin, intranasally and/or using injections.

The “patient” according to the present invention is a mammal, such as a human, which is diagnosed with one of the diseases, disorders or conditions described herein, or is predisposed to at least one type of the diseases, disorders or conditions described herein. The compositions of the present invention can be administered to any mammal that can experience the beneficial effects of the compositions and methods of the invention. Any such mammal is considered a “patient.” Such patients include humans and non-humans, such as humans, domestic and farm animals, and zoo, sports, or pet animals, such as dogs, horses, cats, cows, mice, rats, etc.

The term “curcumin” herein mentioned refers to the principal curcuminoid of turmeric. As described herein, curcumin can be used alone or in combination with other curcuminoids (e.g., demethoxycurcumin or bisdemethoxycurcumin).

The term “curcuminoid(s)” herein mentioned refers to a derivative of curcumin (e.g., demethoxycurcumin, bisdemethoxycurcumin, tetrahydroxycurcumin, Bis-0-Demethyl curcumin (BDMC), or combinations thereof), and/or derivatives of one or more of curcumin, demethoxycurcumin, bisdemethoxycurcumin, tetrahydroxycurcumin or Bis-0-Demethyl curcumin (BDMC).

The term “Eudragit” herein mentioned refers to a polymer or co-polymer having a backbone containing polymethacrylate or methyl methacrylate-based polymer, with such polymer or co-polymer capable of having various different functional side-chains attached to such backbone.

The term “surfactant” herein mentioned refers to an amphipathic molecule, including such molecules that are anionic, cationic, nonionic, synethetic or natural.

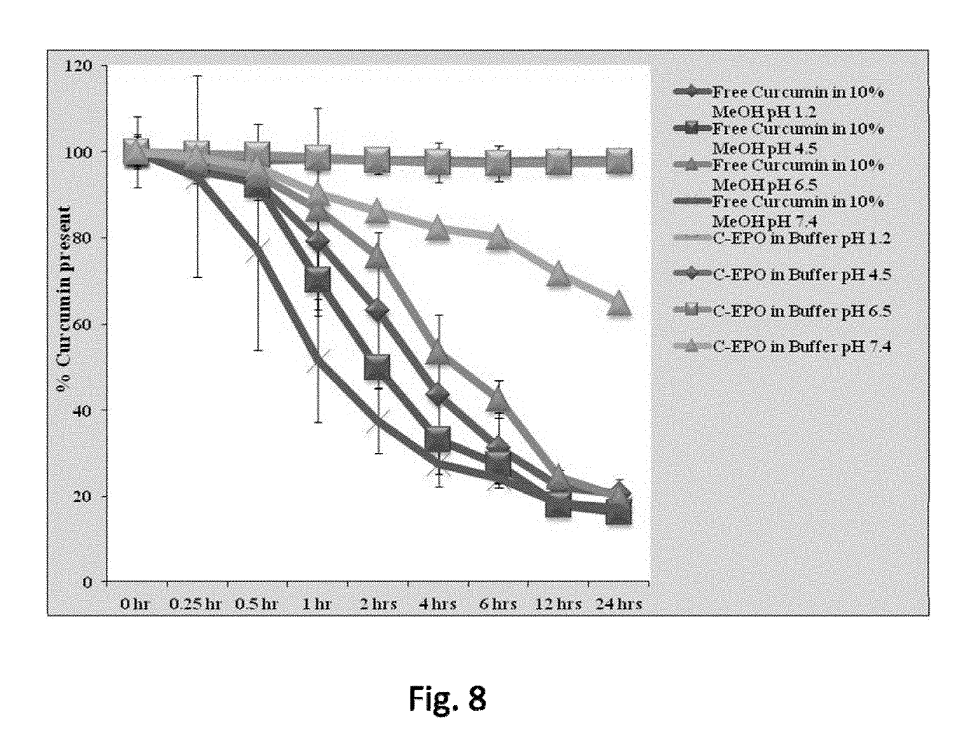

The present invention relates generally to curcumin and/or curcuminoid formulations useful for the treatment of diseases involving cancer, neurodegeneration, inflammation, infection, and immunodeficiency. Specifically, in some aspects, the present invention provides a composition comprising nanoparticles or microparticles loaded with curcumin-polymer complexes having enhanced stability, aqueous solubility and/or bioavailability. As shown in FIG. 8, the presence of a polymer enhances the stability of aqueous soluble curcumin-polymer complexes over a broad pH range, such as pH of 1.2, 4.5, 6.8, 7.4 or higher, compared to free curcumin dissolved in 10% methanol

In some aspects of the present invention, a method of treating patients for diseases involving cancer, neurodegeneration, inflammation, infection and immunodeficiency comprises administering a medicament preparation comprising nanoparticles loaded with curcumin-polymer complexes, the medicament preparation having enhanced bioavailability of the curcumin component.

Curcumin is the active curcuminoid of turmeric and also known as C.I. 75300, diferuloylmethane, or Natural Yellow 3. The systematic chemical name of curcumin is (1E,6E)-1,7-bis(4-hydroxy-3-methoxyphenyl)-1,6-heptadiene-3,5-dione. Curcumin has the following chemical structure:

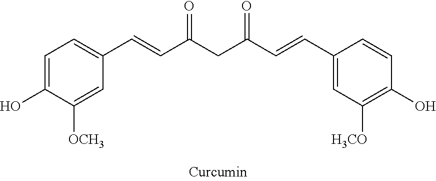 [SMILES: COc1cc(/C=C/C(=O)CC(=O)/C=C/c2ccc(O)c(OC)c2)ccc1O]

Curcumin can exist at least in two tautomeric forms, keto and enol. The -keto form is preferred in solid phase and the -enol form in solution. For example, in acidic solutions (e.g., pH<7.4) curcumin turns yellow, whereas in basic solutions (e.g., pH>8.6), curcumin turns bright red. The biological effects of curcumin involve the inhibition of metabolic enzymes, which can result in antioxidant, anti-inflammatory, and anti-tumor activity. Commercially available curcumin can comprise approximately 77% diferuloylmethane (curcumin), 17% demethoxycurcumin, and 6% bisdemethoxycurcumin. In some cases, curcuminoids (i.e., derivatives of curcumin) can be synthesized to enhance the solubility of curcumin and hence, its bioavailability. For example, other curcuminoids besides curcumin include demethoxycurcumin, bisdemethoxycurcumin, tetrahydroxycurcumin, and Bis-0-Demethyl curcumin (BDMC). In some cases, curcuminoids or other curcumin derivatives can be used independently or in combination to enhance stability, aqueous solubility, bioavailability and/or therapeutic efficacy. As examples of other curcuminoids, the curuminoids demethoxycurcumin and bisdemethoxycurcumin have the following chemical structures:

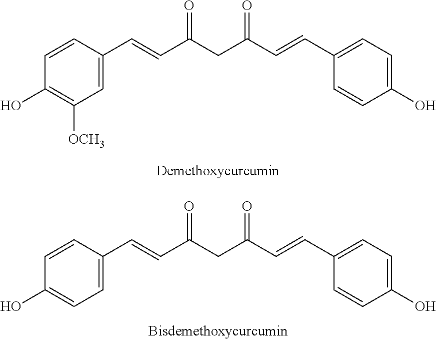 [SMILES: COc1cc(/C=C/C(=O)CC(=O)/C=C/c2ccc(O)cc2)ccc1O ; O=C(/C=C/c1ccc(O)cc1)CC(=O)/C=C/c1ccc(O)cc1]

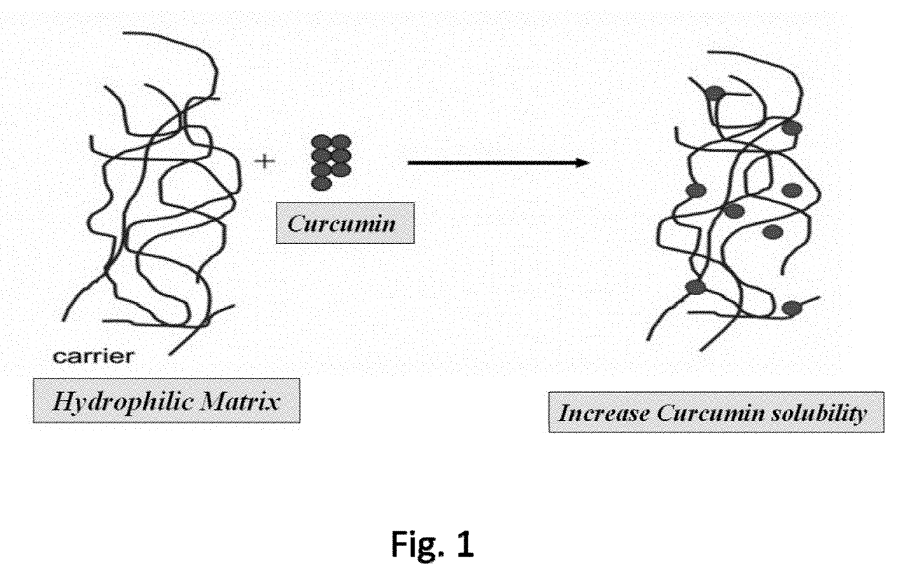

As illustrated in FIG. 1, curcumin can be combined with a polymer (e.g., Eudragit™), which provides a hydrophilic matrix, to form a curcumin-polymer complex that increases the aqueous solubility of curcumin, and in turn, its stability and/or bioavailability. While FIG. 1 illustrates curcumin, the figure is equally representative of other curcuminoids alone or in combination with curcumin that can be combined with a eudragit to form a complex that increases the solubility of the curcumin and/or curcuminoids, and in turn, its stability, aqueous solubility and/or bioavailability.

Eudragit™ copolymers are derived from esters of acrylic and methacrylic acid. In some aspects, Eudragit™ polymers that form a complex with curcumin and/or other curcuminoids according to certain aspects of the present invention comprise a polyacrylate or polymethylacrylate backbone in addition to functional groups which provide unique physiochemical properties (e.g., solubility at different pHs). In some aspects, a Eudragit™ polymers comprising a polyacrylate or polymethacrylate backbone and an anionic, cationic, or neutral functional group/copolymer can enhance the stability, aqueous solubility and/or bioavailability of curcumin and/or other curcuminoids.

For example, the Eudragit™ copolymer can comprise Eudragit® EPO, available from Evonik Industries, which is a cationic copolymer based on dimethylaminoethyl methacrylate, butyl methacrylate, and methyl methacrylate. In some other aspects, the eudragit copolymer can comprise Eudragit® S-100, available from Evonik Industries, which is an anionic copolymer based on methacrylic acid and methyl methacrylate. The Eudragit™ copolymers Eudragit® EPO and Eudragit® S-100 have the following chemical structures:

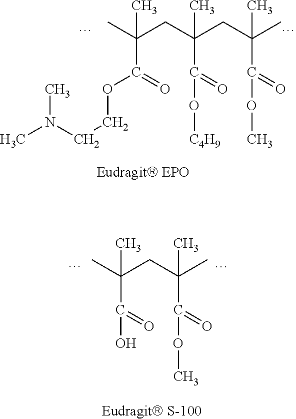

For example, the curcumin formulation of the present invention can include Eudragit EPO® comprising pH-dependent cationic copolymers soluble in gastric fluid or tissues (e.g., stomach) up to a pH of about 5.0. Additionally, for example, the curcumin formulation of the present invention can include Eudragit S100® comprising pH-dependent anionic copolymers soluble in intestinal fluid or tissues (e.g., ileum and colon) ranging between approximately pH 6.5 to approximately 7.5. In still other aspects, the curcumin formulation of the present invention can include curcumin-polymer complexes having various Eudragit™ polymers, such that the curcumin formulation has additional or synergistic benefits. One of ordinary skill in the art shall appreciate that other polymers can be chosen depending on the pH of the specific tissue or tissues targeted for curcumin and/or other curcuminoid therapy.

For example, the complexation of curcumin and two or more polymers (e.g., Eudragit® S100 and Eudragit® EPO) may be more beneficial for inflammatory bowel disease, such that the curcumin-polmer (Eudragit® EPO) complexes will dissolve in the stomach and curcumin will be absorbed and will be delivered to the target colon tissue through blood supply. However, curcumin-polymer (Eudragit® S100) complexes will not dissolve in any part of the intestine until such complexes reach the colon (pH-7.0) and deliver curcumin locally at the colon site through lumen. Thus, by delivering curcumin both systemically through blood supply and locally at the lumen will be more beneficial to the respective patient.

In some embodiments, the polymer can be chosen from the class of Eudragit™ copolymers Eudragit® E, available from Evonik Industries, which are a cationic copolymer based on dimethylaminoethyl methacrylate, butyl methacrylate and methyl methacrylate having a pendant tertiary amine group, which can enhance curcumin aqueous solubility at a pH up to above 5.0 (i.e., the stomach).

In some other embodiments, the polymer can be chosen from the class of Eudragit™ copolymers Eudragit® S, also available from Evonik Industries, which are anionic copolymers based on methacrylic acid and methyl methacrylate having a carboxylic acid group, which can enhance curcumin aqueous solubility at pH of about 7.0 and above (i.e., lower intestine).

In some other embodiments, it is contemplated that the polymer can be chosen from the class Eudragit™ copolymers Eudragit® L, also available from Evonik Industries, which contain an anionic copolymers based on methacrylic acid and ethyl acrylate, which can enhance curcumin aqueous solubility.

In some other embodiments, it is contemplated that the polymer may be chosen from the class of Eudragit™ copolymers Eudragit® R, also available from Evonik Industries, which is a copolymer of ethyl acrylate, methyl methacrylate and a low content of methacrylic acid ester with quaternary ammonium groups. These polymers are time controlled, pH independent. Examples include Eudragit® RL 100, Eudragit® RL PO, Eudragit® RL 30 D, Eudragit® RL 12,5, Eudragit® RS 100, Eudragit® RS PO, Eudragit® RS 30 D, and Eudragit® RS 12,5.

In some other embodiments, it is contemplated that the polymer can be chosen from the class of Eudragit™ copolymers Eudragit® N, also available from Evonik Industries, which is a neutral copolymer based on ethyl acrylate and methyl methacrylate. These polymers are time controlled, pH independent. Examples include Eudragit® NE 30 D, Eudragit® NE 40 D and Eudragit® NM 30 D.

The foregoing examples provide a non-exhaustive list of polymers that are contemplated to form complexes with curcumin and/or other curcuminoids according to certain aspects of the present invention. A representative list of polymers that can form a complex with curcumin and/or other curcuminoid with potential target tissue dissolution and the corresponding pH is provided in Table 1.

TABLE 1
Examples of polymers and their corresponding approximate
pH ranges, target tissues, and chemical structure.

| | Approximate | |
|---|---|---|
| Polymer | pH | Target Tissue |
| Eudragit ® E 100 | 1-5 | Stomach |
| Eudragit ® E 12.5 | 1-5 | Stomach |
| Eudragit ® EPO | 1-5 | Stomach |
| Eudragit ® L-30 D-55 | >5.5 | Duodenum |
| Eudragit ® L 100-55 | >5.5 | Duodenum |
| Eudragit ® L 100 | 6-7 | Jejunum |
| Eudragit ® L 12.5 | 6-7 | Jejunum |
| Eudragit ® S 100 | >7.0 | Ileum, Colon |
| Eudragit ® S 12.5 | >7.0 | Ileum, Colon |
| Eudragit ® FS 30 D | >7.0 | Ileum, Colon |
| Eudragit ® RL 100 | Independent - time | |
| | released | |

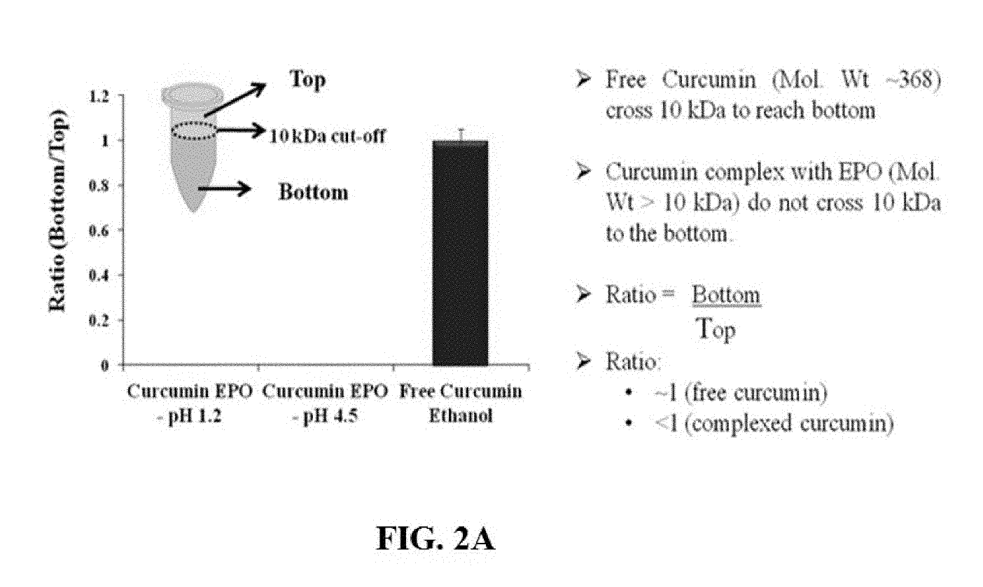

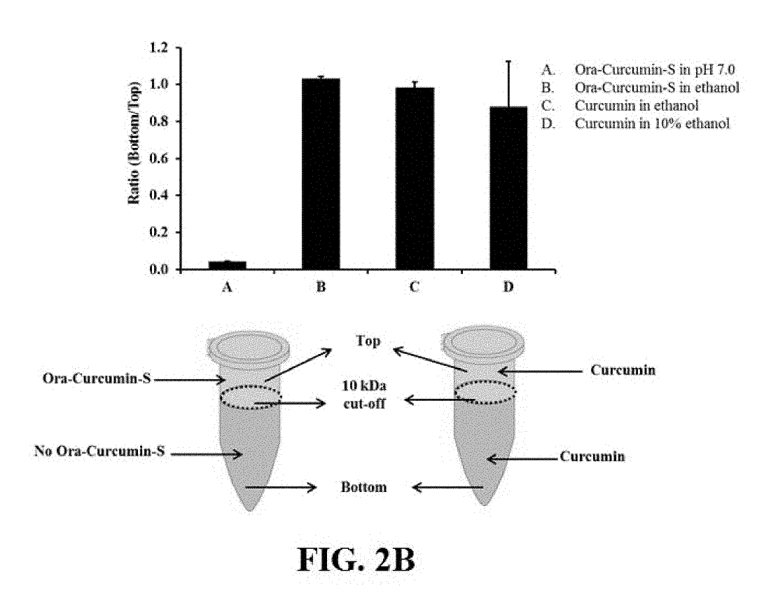

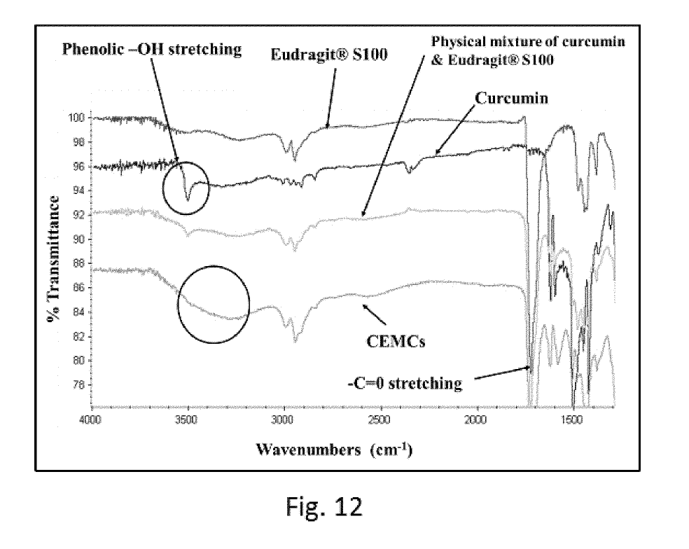

In some aspects, a polymer can form a complex with curcumin and/or one or more curcuminoids based on intermolecular interactions (e.g., hydrophobic interactions, hydrogen bonding or polar bonding), which enhances the bioavailability of curcumin and/or the one or more curcuminoids. As illustrated in FIG. 2, in one embodiment of the present invention, curcumin exists in complexation with a polymer (Eudragit EPO®) at pHs 1.2 and 4.5, which prevents it from crossing the 10 kDa filter. As also shown in FIG. 2, in the absence of the polymer, free curcumin passes through the 10 kDa filter and precipitates. According to some aspects, the methacrylic acid backbone of some polymers can be the basis of the intermolecular interaction with curcumin, in which case, the interaction would be independent of the cationic or anionic copolymer of the polymer component. In other aspects, the basis of the complex formation can be an ionic interaction involving the cationic or anionic copolymers with phenoloc hydroxyl group of the curcumin as demonstrated in FIG. 12.

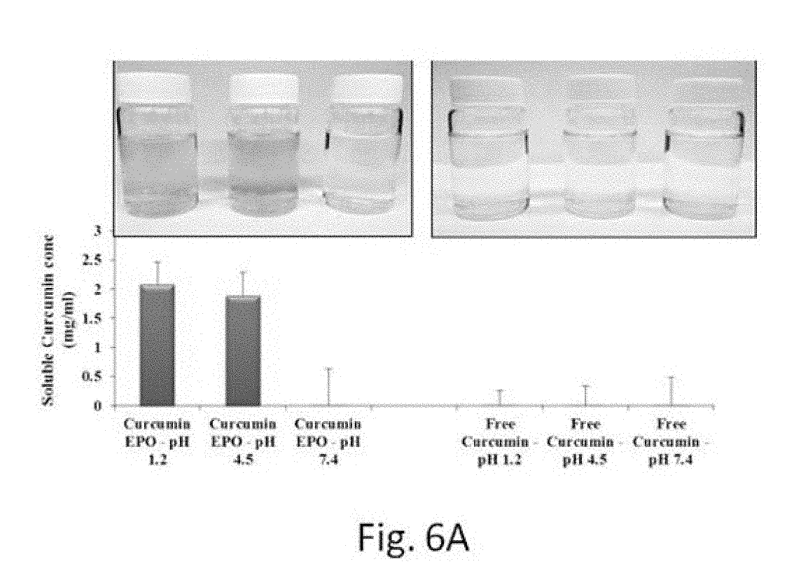

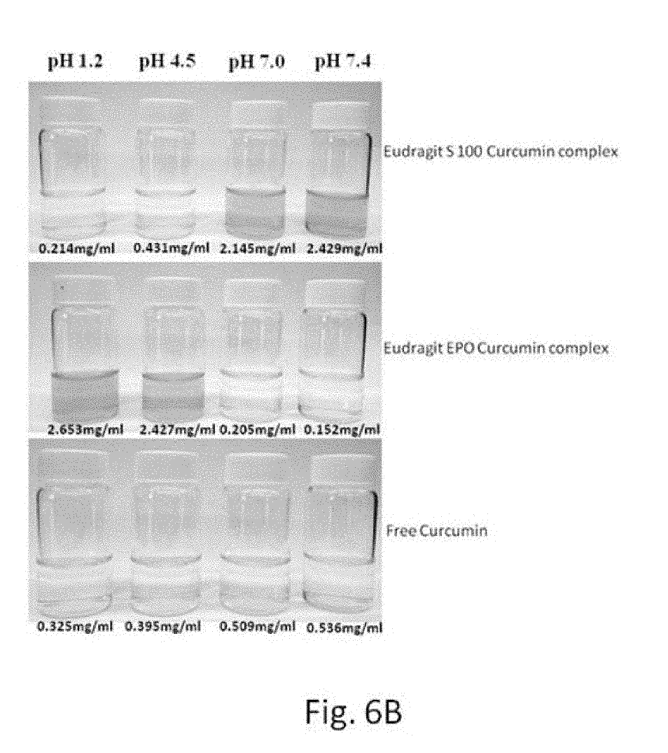

In some aspects, the formation of curcumin-polymer complexes with enhanced bioavailability according to certain embodiments of the present invention involves the use of solubilization and precipitation techniques. As shown in FIGS. 6A and 6B, curcumin solubility is enhanced due to complexation with the polymer component. For example, as shown in FIGS. 6A and 6B, curcumin solubility is enhanced at pH of about 1.2 and pH of about 4.5 (i.e., yellow color) when complexed with Eudragit® EPO polymer as compared to free curcumin. Additionally, as shown in FIG. 6B, curcumin solubility can also be enhanced at pH 7.0 and pH 7.4 when complexed with Eudragit® S100 polymer (top panel) as compared to free curcumin. Depending on the polymer used to form the complex with curcumin and/or curcuminoids, fluids or tissues with different pHs can be differentially targeted for curcumin therapy provides unique tissue specificity to deliver soluble curcumin or curcuminoids. For example, Eudragit S100® can be used as the polymer to complex with curcumin in order to deliver curcumin to the ileum and colon, while Eudragit® EPO can be used as the polymer to complex with curcumin to deliver curcumin to the stomach.

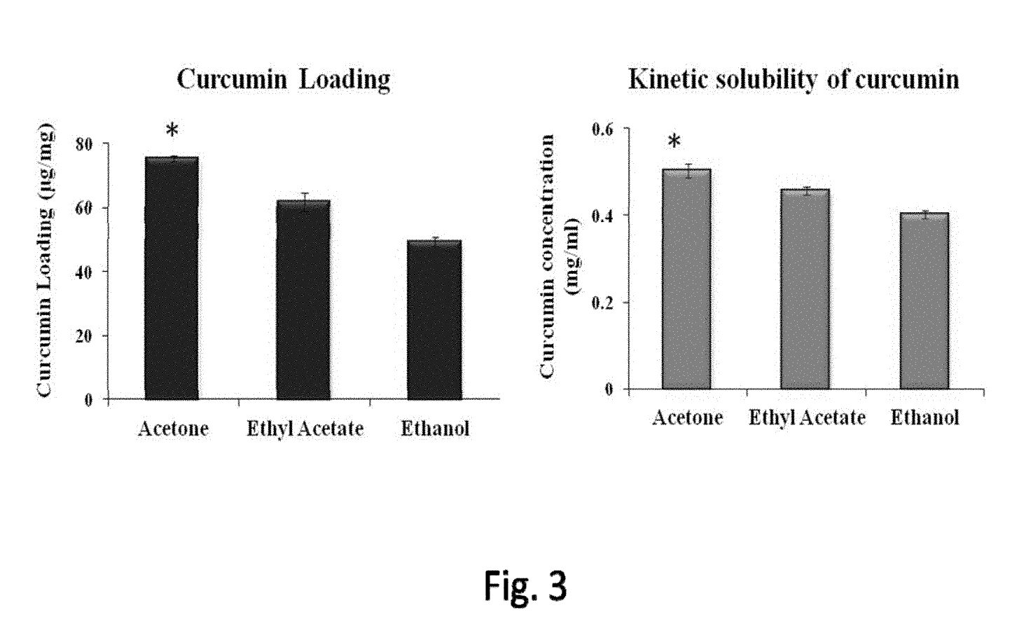

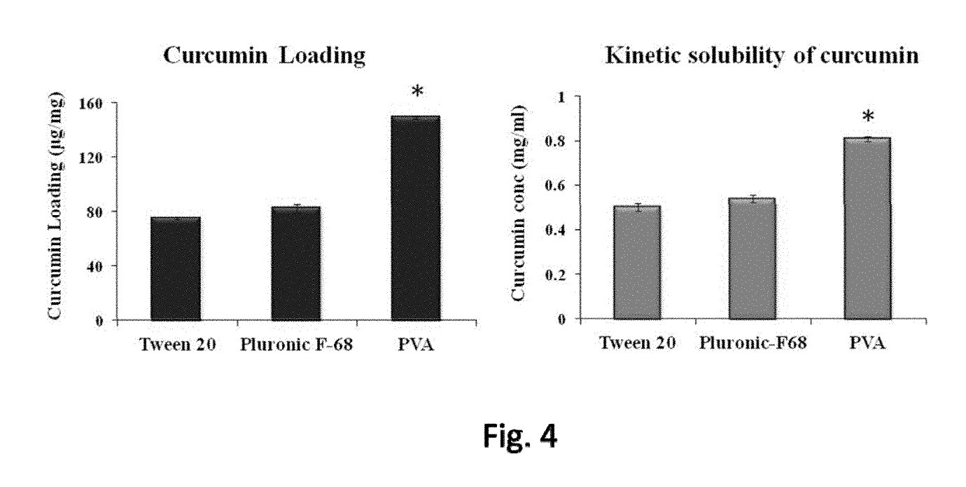

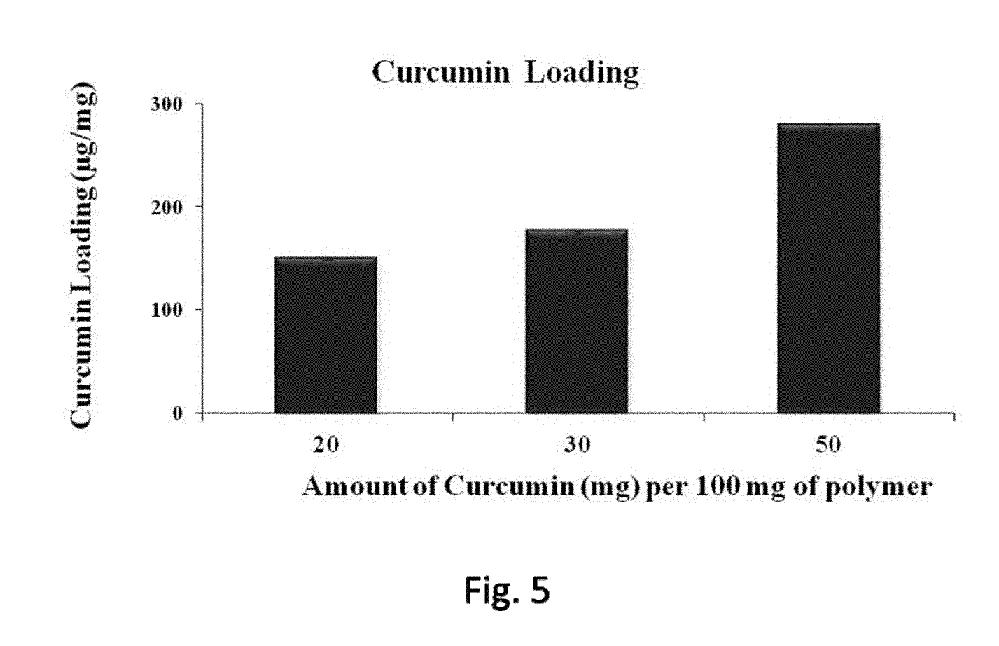

In some aspects, curcumin and a polymer component can be dissolved and then added to an aqueous solution containing a surfactant or a stabilizer, such as shown in FIGS. 3 and 4. In some cases, this process can be performed under other co-precipitation techniques. The resulting particle complexes can be collected and analyzed for the amount of curcumin present in the particles. As shown in FIG. 5, at least 50 mg of curcumin can be added per 100 mg of the Eudragit™ polymer. In some cases, different formulation and process parameters (e.g., type and concentration of organic solvent or surfactant used, curcumin to polymer ratio, etc.) can be used in order to alter the formulation to obtain increased or decreased loading and/or increased or decreased solubility.

As shown in FIG. 3, the use of different organic solvents alter the solubility of the curcumin-polymer complexes. In some cases, as shown in FIG. 4, the particle size can be altered by changing the surfactant type (e.g., Tween-20, Pluronic F68, or polyvinyl alcohol) and the surfactant concentration in the preparation. In some embodiments, the surfactant concentration is between 1% and 20% w/v. In some embodiments, the surfactant concentration is between 1% and 3% w/v. In some embodiments, the surfactant concentration is between 1% and 5% w/v. In some embodiments, the surfactant concentration is between 3% and 5% w/v. In some embodiments, the surfactant concentration is between 5% and 10% w/v. In some embodiments, the surfactant concentration is 1%, 2%, 3%, 4%, 5%, 6%, 7%, 8%, 9%, 10%, 15%, or 20% w/v.

The amount of curcumin accumulation present in the particles can be enhanced by changing the curcumin (and/or curcuminoid)-polymer ratio. In some embodiments, the ratio of curcumin and/or curcuminoid to polymer component is 1:0.1, 1:0.5, 1:1, 1:2, 1:3, 1:4, 1:5, 1:6, 1:7, 1:8, 1:9, 1:10, 1:15, 1:25 or 1:50.

Other parameters can also be altered to change the size of the particles, including the sonication energy or vigorous stirring time. For example, particle size can range from approximately 10 nm to 5000 nm, depending on the solvent and surfactant used during the particle formation process, as well as other experimental parameters, such as shown in FIG. 2. In some embodiments, the particles can be between approximately 10 nm and 50 nm. In some embodiments, the particles can be between approximately 51 nm and 100 nm. In some embodiments, the particles can be between approximately 101 nm and 200 nm. In some embodiments, the particles can be between approximately 201 nm and 300 nm. In some embodiments, the particles can be between approximately 301 nm and 400 nm. In some embodiments, the particles can be between approximately 401 nm and 500 nm. In some embodiments, the particles can be between approximately 501 nm and 1000 nm. In some embodiments, the particles can be between approximately 1001 nm and 2000 nm. In some embodiments, the particles can be between approximately 2001 nm and 3000 nm. In some embodiments, the particles can be between approximately 3001 nm and 4000 nm. In some embodiments, the particles can be between approximately 4001 nm and 5000 nm. In some embodiments, the particles can be between approximately 10 nm and 500 nm. In some embodiments, the particles can be between approximately 10 nm and 200 nm. In some embodiments, the particles can be between approximately 201 nm and 401 nm. In some embodiments, the particles can be between approximately 210 nm and 350 nm.

In some aspects, the curcumin or curcuminoid, either alone or in combination, can be formulated by techniques including, but not limited to, nano/micro precipitation, which can be carried out by methods such as sonication, emulsification, solvent titration, milling, spray drying, solid dispersion, hot-melt extrusion, freeze drying methods, or Supercritical Fluid Technology.

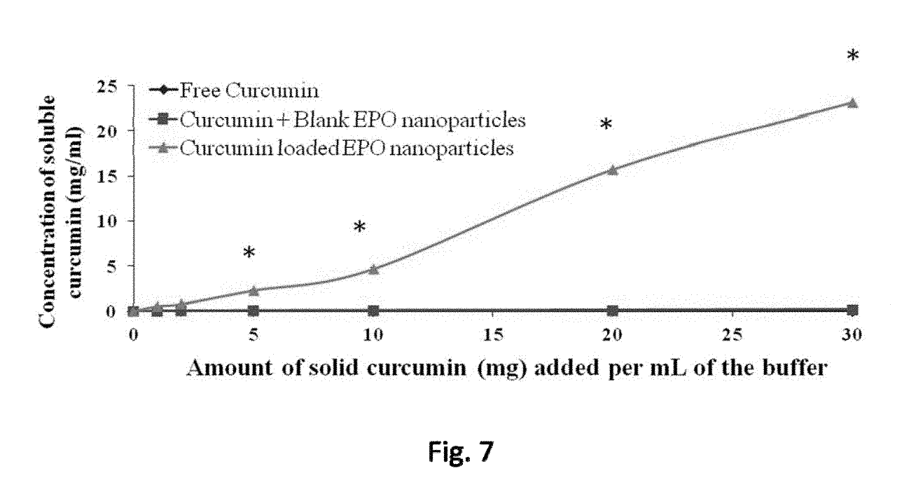

In some aspects, blank nanoparticles or microparticles lacking curcumin can be used to determine the relative solubility and bioavailability of curcumin by comparing them with particles containing curcumin-polymer complexes. For example, blank nanoparticles can be produced alongside nanoparticles containing curcumin-polymer complexes according the methods described above, except that curcumin will not be included in the formulation of the blank nanoparticles. As illustrated in FIG. 7, by physically including the polymer and stabilizer through blank particles with curcumin did not enhanced its aqueous solubility. The curcumin or curcuminoids must to be in complexation with a polymer (precipitated together) for improved solubility observed as shown in the FIG. 7.

In some aspects, therapeutically effective curcumin formulations can comprise an adjuvant that delays or inhibits curcumin metabolism. In some cases, compounds that inhibit P-glycoprotein and glucuronidation can be used to effectively inhibit curcumin metabolism. For example, piperine can be included in the formulation of a composition comprising curcumin-polymer complexes of the present invention to enhance the therapeutic effects of curcumin. Piperine is an alkaloid that can be extracted from black pepper. It is also known as piperidine, piperoylpiperidine, or by its chemical name, 5-(3,4-methylenedioxyphenyl)-2,4-pentadienoyl-2-piperidine. Piperine inhibits the action of certain enzymes involved in the metabolism and transport of xenobiotics and metabolites, and enzymes involved in drug metabolism (e.g., CYP3A4 and P-glycoprotein). By inhibiting drug metabolism, piperine may increase the bioavailability of various compounds and alter the effectiveness of some medications. For example, piperine may enhance the bioavailability of curcumin by 2000% in humans. Delaying the metabolism of curcumin using piperine enhances its therapeutic effects by enabling it to persist longer in a patient's body or penetrate more deeply into target tissues. In some embodiments, the dose of piperine used in the present invention can be at least about 2.5 mg/day. In some embodiments, the dose of piperine can be between from about 1 to about 100 mg/day, or from about 10 to about 20 mg/day. In some embodiments, the source is piper longum derived from black pepper, which comprises at least about 90% piperine.

In some aspects, the formulation of curcumin-polymer complexes of the present invention can comprise other adjuvants to enhance the therapeutic effects of curcumin, including but not limited to, genistein, EGCG (epigallocatechin-3-gallate), vanillin, gingerol, capsaicin or any combinations thereof. In other aspects, the formulation of a composition comprising curcumin-polymer complexes can further comprise other excipients or inactive components to enhance the therapeutic effects of curcumin, including but not limited to, a-lipoic acid, omega3/6 fatty acids, fish oil, vitamin B1, vitamin B6, vitamin D, vitamin B12, folate, vitamin C and/or vitamin E, or any combinations thereof. In other aspects, the formulation of a composition comprising curcumin-polymer complexes can further comprise other therapeutic drugs such as aspirin, salicylic acid, chemotherapeutic drugs, anti-inflammatory drugs, ant-Alzheimer's disease dugs and thereof. In another aspect, the formulation of a composition comprising curcumin-polymer complexes can further comprise other components to enhance the therapeutic effects of curcumin, including but not limited to, cream bases and emulsifiers such as light liquid paraffin, PEG, water washable bases such as cetyl alcohol, stearic acid, stearyl alcohol, glycerol monostearate, lanolin, glycerin and others and solid emulsifiers/nonionic surfactants such as Acconon, polyethylene glycol (PEG 200), glyceryl monosterate (GMS), polyethylene glycol (PEG 400) and Cetyl alcohol (CA) and Tween 80, preservatives such as methyl, ethyl or propyl parabens or bronidox, emollient such as Isopropyl myristate for ready absorption into the skin, collagen for maintaining the skin moisture and to give firmness, other flavoring agents such as lavender oil and antiseptic agents such as 2-phenyl ethanol. In another aspect, the formulation of a composition comprising curcumin-eudragit complexes invention can further comprise pharmaceutically, nutraceutically or dietically acceptable anti-inflammatory, anti-psoriatic, antioxidant, anti allergic, antiviral, antibacterial, anti-cancer, anti-neurodegeneration, and anti-angiogenic agents.

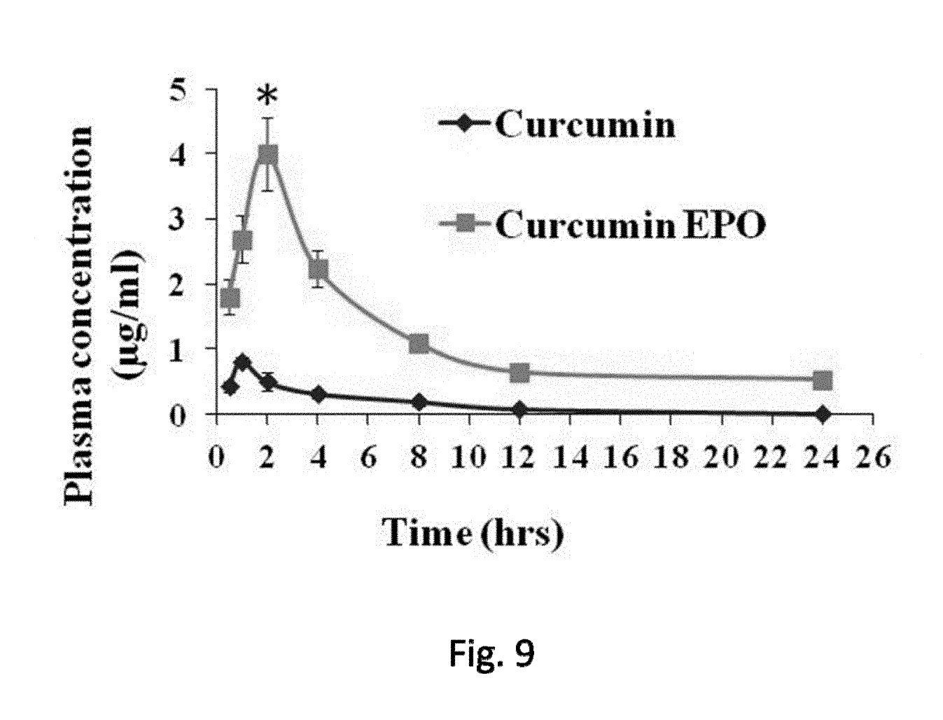

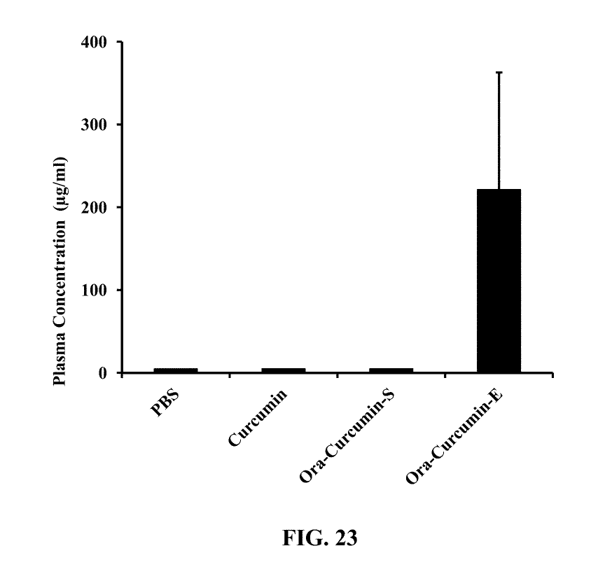

In some aspects, the invention provides a method of treating patients for diseases such as cancer, neurodegeneration, inflammation, infection and immunodeficiency comprising the administration of a composition comprising curcumin-polymer complexes having enhanced bioavailability or selective delivery of soluble curcumin to specific regions of GIT based on the local pH. The curcumin-polymer complexes of the present invention can enhance the solubility of curcumin up to 20,000 times or even more compared to free curcumin or could enhance the selective delivery of bioactive curcumin to various regions of the GIT. In some aspects, the solubility of the curcuminoid component of the medicament preparation is enhanced between about 10 and about 100 times, in some aspects between about 100 and about 500 times, in some aspects between about 500 and about 1000 times, in some aspects between about 1000 and about 5000 times, in some aspects between about 5000 and 10000 times, in some aspects between about 10000 and 15000 times, and in some aspects between about 15000 and 20000 times. As shown in FIGS. 9 and 23, the complexation of curcumin with Eudragit®-EPO polymer enhances the bioavailability of curcumin in the blood of mice, as compared to free curcumin.

In some embodiments, a composition comprising curcumin-polymer complexes can be taken orally in the form of a solid (e.g., tablet or pill), a liquid (e.g., solution, suspension or lotion), or semisolid (e.g., gel, cream or ointment). It some embodiments, these compositions be delivered orally and the components be prepared for ingestion in a manner that makes the composition available in therapeutically effective amounts. As such, they may be prepared as water soluble compositions, delivered in liquid form, lyophilized, encapsulated, or in a manner suitable for time release, delayed release or enteric delivery, or any manner typically used for orally delivered pharmaceuticals, nutraceuticals or vitamins, or combined with foods or other normally ingested products. However, the present invention is not limited to oral delivery, as the compositions set forth herein may also be delivered by nasal spray, inhalation techniques, transdermally, transmucossally, ocularly, by suppository, injected, or by intravenous methods. For example, in other embodiments, the compositions comprising curcumin-polymer complexes can be injected in to the patient's body systemically, or injected into a specific target tissue. In other embodiments, curcumin-polymer complexes can be applied topically to the patient's skin to treat specifically an applied area, or generally to provide more widespread or systemic therapy. In other embodiments, curcumin-polymer complexes can be delivered to various body's orifices, eyes, nose, ears, mouth, urethral, vaginal or rectal

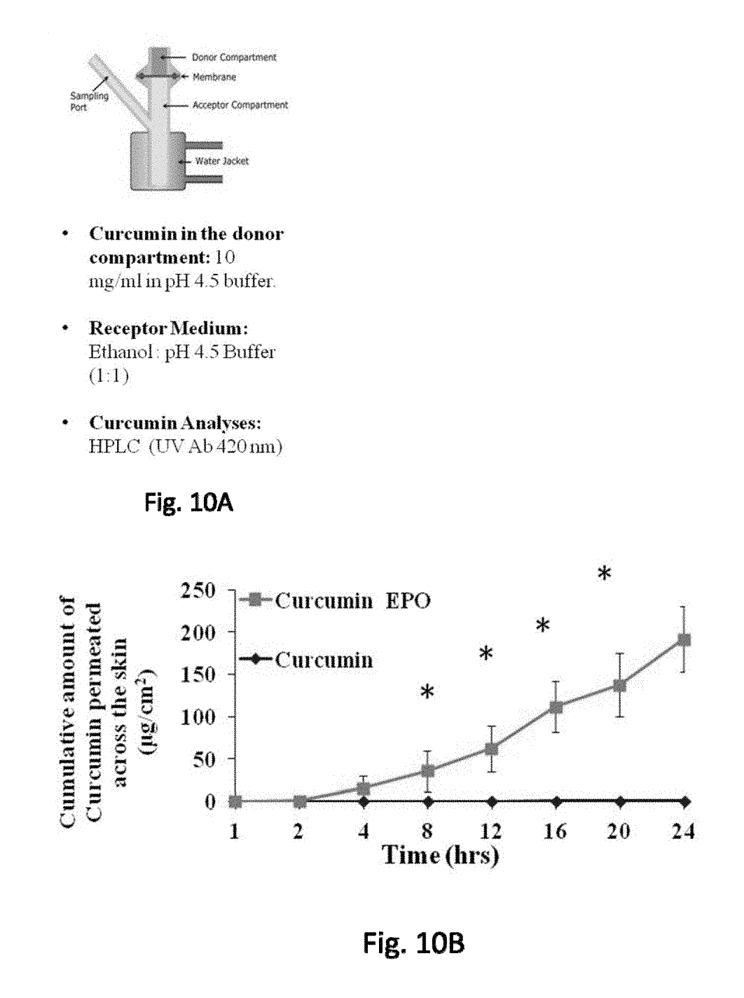

As shown in FIG. 10B, the amount of curcumin transported across the skin tremendously increased when curcumin is delivered as a molecular complex with a polymer. In other embodiments, the topical formulation of curcumin-polymer complexes is in a dosage form selected from the group consisting of semisolid dosage forms, ointments, creams, solutions, mouthwash, skin patches, eye drops, medicated sticks, lozenges, pastes, toothpastes, gels, lotions, or suppositories.

In some embodiments, the compositions comprising curcumin-polymer complexes of the present invention are to be administered at a dosage of from about 0.1 mg/kg/day to about 1 mg/kg/day. In some embodiments, the compositions of the present invention can be administered in a dosage of about 200 mg/day to about 15,000 mg/day. The dosage to be administered can comprise, for example, curcumin in an amount of from about 1.05 to about 85 mg/kg patient body weight, or from about 8.8 to about 13.4 mg/kg body weight, or from about 11.1 to about 111 mg/kg patient body weight, or from about 88.8 to about 133.2 mg/kg patient body weight. The dosage to be administered can also comprise, for example, piperine in an amount of from about 0.01 to about 1.0 mg/kg patient body weight, or from about 0.09 to about 0.9 mg/kg patient body weight or from about 0.09 to about 0.11 mg/kg patient body weight, or from about 0.7 to about 1.1 mg/kg patient body weight.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In addition, the materials, methods, and examples are illustrative only and not intended to be limiting. Other features and advantages of the invention will be apparent from the following detailed description, and from the claims.

### EXAMPLES

### Formation of Curcumin-Polymer Complexes

### Example 1

### Formation of Nanoparticles Loaded with Curcumin-Eudragit® EPO Complexes

Curcumin and piperine-loaded Eudragit® EPO nanoparticles were prepared using a nanoprecipitation method. During the preparation, either the combination of curcumin and the Eudragit™ polymer, or the combination of curcumin, piperine and the Eudragit™ polymer were dissolved in an organic solvent. The solution was added drop-wise to an aqueous solution containing a surfactant, under sonication. The resulting dispersion was stirred until complete evaporation of the organic solvent. The nanoparticles/microparticles were collected by centrifugation, lyophilized, and stored at 4° C. The amount of curcumin and piperine present in the nanoparticles (loading) was determined by dissolving the particles in ethanol and quantifying the amounts of curcumin (at 262 nm) and piperine (at 342 nm) by HPLC with UV detector. Different formulation and process parameters (e.g., type and concentration of organic solvent or surfactant used, curcumin to eudragit polymer ratio, etc.) were tested in order to optimize the formulation to obtain higher loading and better solubility. The particle size was altered by changing the surfactant type (e.g., Tween-20, Pluronic F68, or polyvinyl alcohol) and the surfactant concentration (1, 2, or 3% w/v) in the preparation. The loading of the curcumin in the formulation (amount of curcumin/mg of formulation) was enhanced by changing the curcumin-polymer ratio (1:5, 1:3, or 1:2). Other parameters that could be altered to change the size of the particles included the amount of energy used during sonication and total sonication time.

### Example 2

### Physiochemical Characterization of Nanoparticles Loaded with Curcumin-Polymer (Eudragit® EPO) Complexes Prepared Using Three Different Solvents

Nanoparticles loaded with curcumin-polymer (Eudragit® EPO) complexes were prepared using tween-20 as surfactant in three different solvents (acetone, ethyl acetate and ethanol) and 20 mg of curcumin. Curcumin apparent solubility was measured by dispersing Eudragit® EPO particles equivalent to 2 mg of curcumin in 1 ml of pH 1.2 buffer and incubated at 37 C for 4 hrs with 100 rpm shaking. The curcumin dissolved after 4 hrs was analyzed spectrophotometrically. The curcumin loading and curcumin kinetic solubility data is summarized in Table 2, and illustrated in FIG. 3, whereby the data represents mean±standard deviation (n=3). P.I. stands for Polydispersity Index, which was not measured because of aggregation of the particles. Loading represents μg of curcumin present in 1 mg of Eudragit EPO particles. The “*” indicates results were found statistically significant (P<0.05) using a Student t-test.

TABLE 2
Physiochemical characterization of nanoparticles loaded
with curcumin-polymer (Eudragit ® EPO)
complexes prepared using three different solvents.

| | | | | | Curcumin |
|---|---|---|---|---|---|
| | | | | Curcumin | kinetic |
| Sample | Sol- | | | loading | solubility |
| No. | vents | Particle size | P.I. | (μg/mg) | (mg/ml) |
| 1 | Acetone | aggregation | NA | 75.17 ± 0.89 * | 0.51 ± 0.02 * |
| 2 | Ethyl | aggregation | NA | 61.70 ± 2.92 | 0.45 ± 0.01 |
| | Acetate | | | | |
| 3 | Ethanol | aggregation | NA | 49.28 ± 1.51 | 0.40 ± 0.02 |

### Example 3

### Physiochemical Characterization of Nanoparticles Loaded with Curcumin-Polymer (Eudragit® EPO) Complexes Prepared Using Three Different Surfactants

Nanoparticles loaded with curcumin-polymer (Eudragit® EPO) complexes in acetone were prepared using tween-20, Pluronic F-68 or PVA as a surfactant and 20 mg of curcumin. Curcumin apparent solubility was measured by dispersing Eudragit® EPO particles equivalent to 2 mg of curcumin in 1 ml of pH 1.2 buffer and incubated at 37 C for 4 hrs with 100 rpm shaking. The curcumin dissolved after 4 hrs was analyzed spectrophotometrically. The curcumin loading and curcumin kinetic solubility data is summarized in Table 3, and illustrated in FIG. 4, whereby the data represents mean±standard deviation (n=3). P.I. stands for Polydispersity Index. “NA” suggests particle size and polydispersity index were not measured because of aggregation. Loading represents μg of curcumin present in 1 mg of Eudragit® EPO particles. The “*” indicates results were found statistically significant (P<0.05) using a Student t-test.

TABLE 3
Physiochemical characterization of nanoparticles loaded with curcumin-polymer
(Eudragit ® EPO) complexes prepared using three different surfactants.

| Sample | | | | Curcumin loading | Curcumin kinetic |
|---|---|---|---|---|---|
| No. | Surfactants | Particle Size | P.I. | (ug/mg) | solubility (mg/ml) |
| 1 | Tween-20 | aggregation | na | 75.17 ± 0.89 | 0.51 ± 0.02 |
| 2 | Pluronic F-68 | 343.44 ± 33.27 | 0.57 ± 0.17 | 82.52 ± 2.92 | 0.54 ± 0.01 |
| 3 | PVA | 213.27 ± 12.21 | 0.31 ± 0.12 | 149.24 ± 0.99 * | 0.81 ± 0.01 * |

### Example 4

### Physiochemical Characterization of Nanoparticles Loaded with Curcumin-Polymer (Eudragit® EPO) Complexes Prepared Using PVA as the Surfactant and Different Amounts of Curcumin

Nanoparticles loaded with curcumin-polymer (Eudragit® EPO) complexes were prepared using different total amounts of curcumin, with PVA as the surfactant. Curcumin apparent solubility was measured in pH 1.2 buffer at 37 C for 4 hrs with 100 rpm shaking. The curcumin dissolved after 4 hrs was analyzed from different samples spectrophotmetrically. Data represents mean±standard deviation (n=3). P.I. stands for Polydispersity Index. Loading represents μg of curcumin present in 1 mg of Eudragit EPO nanoparticles. As provided by the data in Table 4, and illustrated in FIG. 5, at least up to 50 mg of curcumin can be added per 100 mg of the Eudragit™ polymer.

TABLE 4
Physiochemical characterization of nanoparticles loaded with
curcumin-polymer (Eudragit ® EPO) complexes prepared
using PVA as the surfactant and different amounts of curcumin.

| Sample | | | | Curcumin loading |
|---|---|---|---|---|
| No. | Curcumin | Particle size | P.I. | (ug/mg) |
| 1 | 20 mg | 213.27 ± 12.21 | 0.41 ± 0.12 | 149.24 ± 0.99 |
| 2 | 30 mg | 241.32 ± 21.22 | 0.37 ± 0.09 | 175.66 ± 0.88 |
| 3 | 50 mg | 251.32 ± 31.31 | 0.42 ± 0.14 | 278.61 ± 2.72 |

### Example 5

### Verification of the Formation of Curcumin-Polymer (Eudragit® EPO) Complexes

Curcumin-polymer (Eudragit® EPO) complexes were prepared with PVA as the stabilizer using the aforementioned methods. The formation and existence of the complex, even in the solution form, was confirmed by passing the solution of curcumin or curcumin equivalent of Ora-Curcumin-S/Oracurcumin-E through protein concentrator tubes with known molecular weight cut-off (10 kDa) (FIG. 2). Curcumin and curcumin equivalent of Ora-Curcumin-S were dissolved in absolute ethanol, 10% and 0 v/v ethanol in 50 mM phosphate buffer pH 7.0 as shown in the FIG. 2B. The ratio of the concentration of soluble curcumin present in the solution after passing through the 10 kDa cut-off membrane (bottom) to the concentration measured before filtration (top) was compared. Data represent mean±standard deviation (n=3-4). Complex formation with high molecular weight Eudragit® 5100 (˜125 kDa) will prevent curcumin to filter through the membrane with 10 kDa cutoff. Curcumin with a molecular weight of 0.368 kDa, completely passed through a 10 kDA cut-off membrane as a solution. In contrast, curcumin present in Ora-Curcumin-S with Eudragit® 5100 with a molecular weight of ˜125 kDa is did not pass through the 10 kDa membrane in significant amounts. Complex formation with the high molecular weight Eudragit® S100 prevented the CEMC from filtering through the membrane. The study was repeated with Curcumin-Eudragit-EPO complexes with similar results (FIG. 2A).

### Example 6

### Bioavailability of Curcumin-Polymer (Eudragit® EPO) Complexes in Mice

Curcumin-polymer (Eudragit® EPO) complexes were prepared according to the aforementioned methods. Free curcumin (150 mg/kg) or the equivalent amount of curcumin-polymer (Eudragit® EPO) complexes were orally administered to mice. Blood samples were then collected from the mice at 0.5, 1, 2, 4, 8, 12, and 24 hours after initiation of the experiment. Curcumin was extracted from the blood plasma and analyzed via by HPLC with UV absorption at 420 nm, with the results illustrated in FIG. 9 indicating an increased amount of plasma concentration of the curcumin-polymer (Eudragit® EPO) complex compared to free curcumin.

### Example 7

### In-Vitro Bioavailability of Curcumin-Polymer (Eudragit® EPO) Complexes Across Porcine Skin

Curcumin-polymer (Eudragit® EPO) complexes were prepared according to the aforementioned methods, including Example 1. In-vitro skin penetration studies were then performed by topically administering nanoparticles loaded with curcumin-polymer (Eudragit® EPO) complexes to porcine skin using the apparatus illustrated in FIG. 10A. Free curcumin (10 mg/ml) or the equivalent amount of curcumin-polymer (Eudragit® EPO) complexes were added to the donor chamber in pH 4.5 buffer, and the amount of curcumin transported across the skin was determined by HPLC. The amount of curcumin that permeated the skin was measured in μg/cm². Samples were collected at 1, 2, 4, 8, 12, 16, 20, and 24 hours after initiation of the experiment. The cumulative amount of curcumin permeated across the skin measured in μg/cm² is illustrated in FIG. 10B, which shows the free curcumin amount remained relatively flat around 0 while the curcumin-polymer (Eudragit® EPO) complex continued to increase the entire 24 hour period.

### Example 8

### Applications of Curcumin-Polymer Complex to the Intestine and Colon Tissue

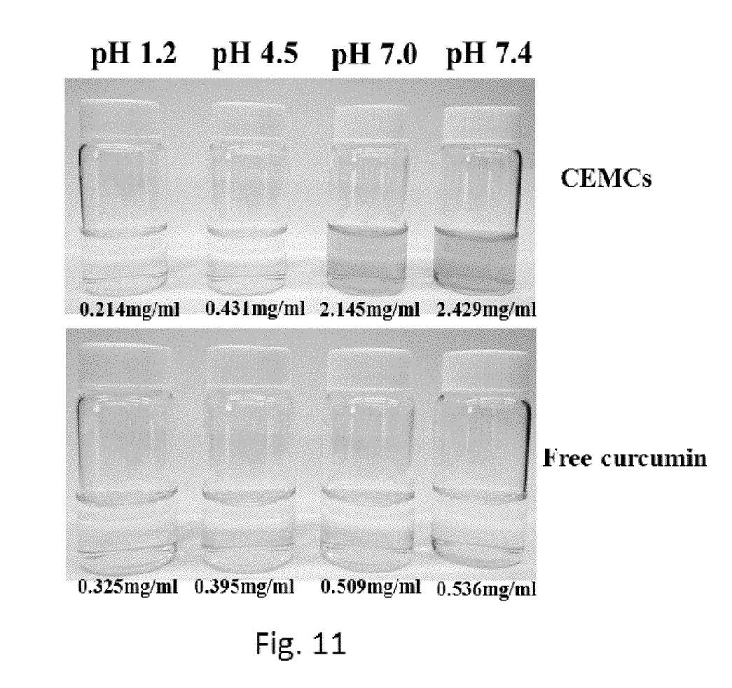

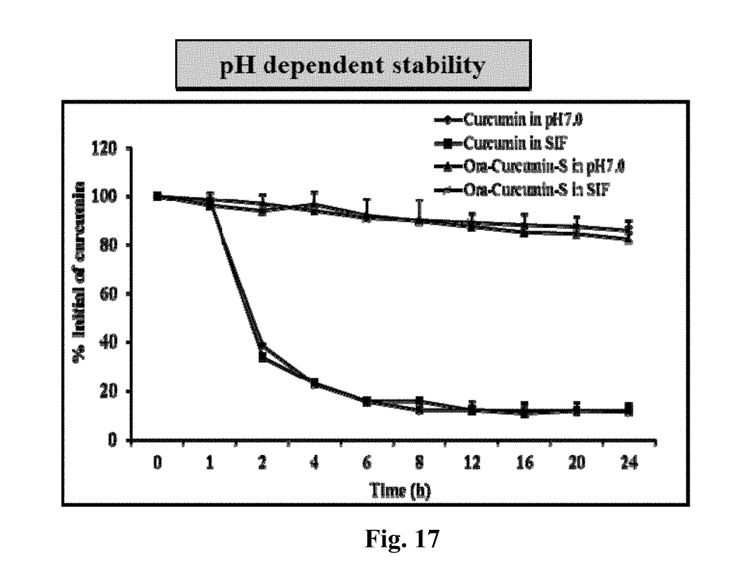

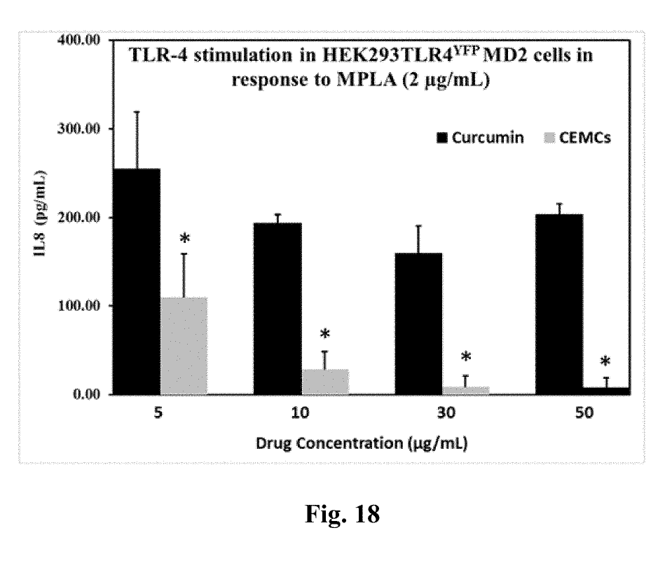

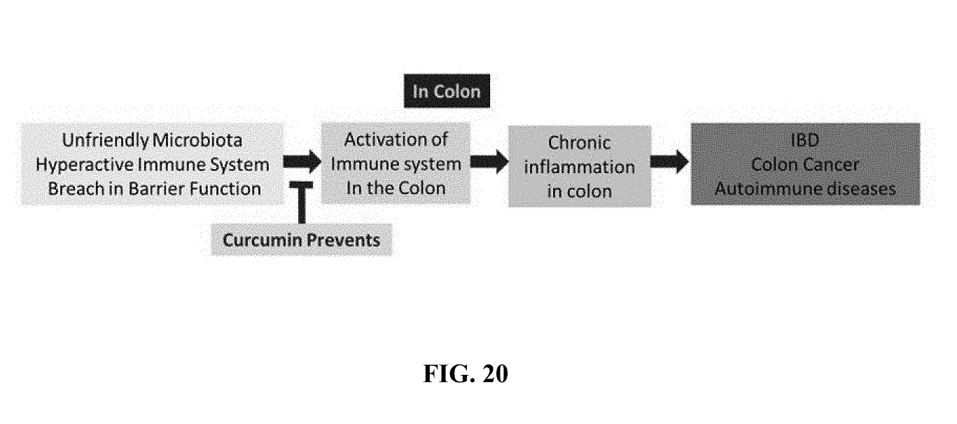

The molecular complexes of curcumin with Eudragit™ polymers according to the present invention were formed to enhance the aqueous solubility and stability of the curcumin. These curcumin-polymer complexes possessed pH-dependent solubility, which is applicable in delivering soluble curcumin to the specific regions of the GI-tract based on the local pH at the region. For example, as shown in FIG. 11, Ora-Curcumin-E is soluble at lower pH (pH<4.5). Therefore, when consumed will be dissolved in the stomach fluids with a pH˜2.0 and provided high concentrations of soluble curcumin for enhanced absorption from the stomach and the intestine into the blood stream (FIGS. 9 and 23). In contrast, Ora-Curcumin-S is soluble at pHs above 6.8 (FIG. 11). Therefore, once consumed orally, Ora-curcumin-S is not expected to dissolve till the GI pH reaches around 6.8, i.e. only closure to colon or large intestine, thereby, significantly reducing the systemic absorption of the drug, while providing highly soluble concentrations locally at the inflammation site of IBD or colorectal cancer (FIG. 23). In addition, curcumin-polymer complexes have better aqueous stability than soluble curcumin as shown in FIGS. 8 and 17. Importantly, these complexes are biologically active to prevent inflammation (FIGS. 18-22). Therefore, the delivery of soluble, stable and biologically potent curcumin to a specific part of the intestine and colon tissue based on local pHs will be significantly beneficial in combating local diseases such as oral mucositis, gastritis, H. Pylori infections, irritable bowel syndrome, inflammatory bowel disease, colo-rectal cancer or system disease (with improved bioavailability) such as Alzheim,er's disease, sepsis, arthritis, cancer etc. For each of the above diseases, a unique polymer can be selected for complexation. GRAS reagents, which are generally regarded as safe for oral consumption for humans, inexpensive and readily available in larger amounts, were employed to translate discoveries to the clinic. Eudragit® S100 polymer provides targeted drug release to the colon after oral consumption. The curcumin-polymer molecular complexes (CEMCs) of the present invention, the Eudragit™ polymer component comprising at least one polymer or co-polymer component having a backbone comprising polymethacrylate or methyl methacrylate, provide improved solubility and stability, reduced systemic side effects due to decreased systemic absorption, high local concentrations of the drug, improved patient compliance because of reduced dosing frequency, and the use of FDA approved agents.

### Cell Lines and Cell Culture

Human origin HEK-TLR-4^{YFP}-MD-2 cell line (NR-9315) was obtained through the BEI resources, ATCC (Manassas, Va., USA). Mouse dendritic cells (DC2.4) are a generous gift from Dr. K. L. Rock (University of Massachusetts Medical Center, Worcester, Mass.). HCT116 (Human colorectal carcinoma) and HT-29 (Human colorectal adenocarcinoma) were purchased from the ATCC (Manassas, Va., USA). All the cell lines were cultured in DMEM-high glucose medium (Thermofisher Scientific, USA) supplemented with penicillin/streptomycin and 10% fetal bovine serum (FBS). A final concentration of 50 μM of β-mercaptoethanol was maintained in the medium for culturing DC2.4 cells. The cells were cultured in a humidified atmosphere of 5% CO₂ at 37° C.

### Formation of Curcumin-Polymer (Eudragit® S100) (Ora-Curcumin-S).

Ora-Curcumin-S was prepared using nanoprecipitation method as previously described. Curcumin and Eudragit® S100 polymer were dissolved in an organic solvent. The solution was then added drop-wise to an acidic aqueous solution containing a 3% w/v of surfactant under constant stirring (400 rpm). The resulting dispersion was stirred until the complete evaporation of organic solvent (˜16 h). Subsequently, the dispersion was centrifuged to collect the formed complexes, freeze-dried and stored in the dark at less than −20° C. until further use. Precaution was taken throughout to protect the curcumin from light and all the experimental procedures were undertaken in dark. Ora-Curcumin-E was also prepared by similar method, except that sterile water was used instead of acidic water to prepare the surfactant/stabilizer solution. The complexes could be prepared with or without the stabilizer or surfactant such as PVA.

### Measurement of (CEMCs)

Curcumin loading (loading represents the micrograms of curcumin present per milligram of curcumin-polymer formulation) was determined by first extracting the curcumin from a known amount of curcumin-polymer complex (5 mg) into 1 ml of dimethyl sulfoxide (DMSO). The amount of curcumin present in the soluble extract was measured at 420 nm using an UV-visible spectrophotometer. A standard curve prepared by using a synthetic curcumin in DMSO waS used to calculate the concentration of the curcumin in the extract. The interference of the other formulation components at 420 nm was ruled out before establishing the spectrophotometric method for analysis.

### Method Optimization

The process of CEMC formation was optimized to enhance curcumin loading and its aqueous solubility by varying the type of organic solvent and surfactants/stabilizer used to prepare the molecular complexes. The ratio of curcumin to the polymer used in the preparation of the molecular complexes was also varied from 1:1, 1:2, 1:3, 1:5, 1:10 and 1:20 to further enhance the curcumin loading. The data for Ora-Curcumin-E is presented in FIGS. 3-5. Acetone as a solvent and PVA as a stabilizer provided the highest loading for Ora-Curcumin-E in the tested formulations.

For Ora-Curcumin-S, the highest curcumin loading up to ˜150 μg/mg was achieved when the curcumin to polymer ratio was 1:2 with acetone+DMSO as an organic solvent and PVA as a surfactant. (Table 5).

TABLE 5
Optimization of loading of curcumin from CEMCs prepared using four different
organic solvents and surfactants. Data represents mean ± standard deviation (n = 3).

| | | | | Curcumin | Apparent |
|---|---|---|---|---|---|
| Sample | | | Ratio | loading | Solubility |
| No. | Solvent | Surfactant | Curcumin:Polymer | (μg/mg) | (mg/ml) |
| 1 | Acetone | Polyvinyl | 1:2 | 115.50 ± 6.01 | 0.539 ± 0.057 |
| | | alcohol (PVA) | | | |
| | | PluronicF68 | | 89.41 ± 9.09 | 0.582 ± 0.014 |
| | | Tween-20 | | 96.57 ± 7.58 | 0.267 ± 0.013 |
| 2 | Ethanol | Polyvinyl | 1:2 | 45.60 ± 1.18 | 0.068 ± 0.051 |
| | | alcohol (PVA) | | | |
| | | PluronicF68 | | 66.38 ± 4.74 | 0.056 ± 0.017 |
| | | Tween-20 | | 131.0 ± 14.60 | 0.373 ± 0.030 |
| 3 | Methanol | Polyvinyl | 1:2 | 50.02 ± 1.81 | 0.234 ± 0.041 |
| | | alcohol (PVA) | | | |
| | | PluronicF68 | | 43.84 ± 0.52 | 0.234 ± 0.029 |
| | | Tween-20 | | 59.21 ± 2.31 | 0.060 ± 0.016 |
| 4 | Acetone + DMSO | Polyvinyl | 1:2 | 152.84 ± 1.55 | 1.093 ± 0.27 |
| | (7 to 3 parts) | alcohol (PVA) | | | |
| | | PluronicF68 | | 140.6 ± 6.51 | 0.653 ± 0.046 |
| | | Tween-20 | | 85.44 ± 7.79 | 0.291 ± 0.099 |

### Measurement of Curcumin Apparent Solubility in Aqueous Buffers

To calculate/estimate/determine the apparent solubility (apparent solubility refers to the solubility measured at 4 h and not the solubility at equilibrium) of curcumin or equivalent amount of curcumin-polymer (Eudragit® 5100) complex were dispersed in pH 1.2, 4.5, 7.0 and 7.4 solution and incubated at 37° C. for 4 h with 100 rpm shaking. After incubation, the samples were centrifuged at 20,000 g, the supernatant filtered through 0.2 μM filter and subsequently analyzed for the concentration of curcumin by using measuring UV absorbance at 420 nm.

As shown in FIG. 11, the curcumin-polymer (Eudragit® S100) complexes were compared to free curcumin in solution. Ora-Curcumin-S was highly soluble at pH of 7.0 and 7.4 and Ora-Curcumin-E had enhanced solubility at pH of 1.2 and 4.5. In contrast, unformulated curcumin was practically insoluble at all pHs

Once the preparation of Ora-Curcumin-S was optimized, the apparent solubility of complexes was investigated in pH 7.0 buffer and incubated at 37° C. for 4 h with 100 rpm shaking, the pH 7.0 buffer representing the pH of the lower part of the intestine/bowel and colon after oral consumption. After incubation, the samples were centrifuged at 20,000 g, the supernatant filtered through 0.2 μM filter and subsequently analyzed for the concentration of curcumin by measuring UV absorbance at 420 nm. The aqueous solubility of curcumin was tremendously enhanced (˜1000 times) when delivered as Ora-Curcumin-S compared to unformulated. Physical addition of curcumin to blank Eudragit® 5100 complexes could not enhance the solubility of curcumin (data not shown). Blank Eudragit® S100 complexes were prepared by precipitating Eudragit® S100 without curcumin in the presence of PVA. This observation suggests that the phenomenon is not in-situ because of the polymer or the surfactant; the complex needs to be formed prior to its addition to aqueous buffers. Similar to loading, the highest apparent aqueous solubility was observed when acetone+DMSO was employed as a solvent and PVA as a surfactant, and with 1:2 drug polymer ratio were selected (1.09±0.27 mg) while preparing Ora-Curcumin-S.

### Measurement of Aqueous Stability of Curcumin Complexes

The aqueous stability of Ora-Curcumin-S was assessed using simulated intestinal fluid TS (BICCA) (pH 6.7-6.9) and at pH 7.0. Curcumin or the curcumin equivalent of Ora-Curcumin-S were solubilized in simulated intestinal fluid TS and pH 7.0 buffer containing 10% methanol and incubated at 37° C. At different time intervals (0, 1, 2, 4, 6, 8, 12, 16, 20, 24 h), the samples were collected, centrifuged (20,000 g) and the supernatant was passed through 0.2 μM filter. The amount of soluble and stable curcumin in the filtrate was determined by using a UV-Visible spectrophotometer at 420 nm.

Once the concern related to the solubility of curcumin has been addressed, another major bottleneck in the oral delivery of curcumin is its aqueous stability, which was assessed for 24 h at pH 7.0 or the pH of simulated intestinal fluid (pH 6.7-6.9) along with 10% methanol. The reason to use 10% methanol was to reach optimum solubility of curcumin at initial stages for the detection of curcumin even after 80% of degradation. Addition of 10% methanol did not alter the complex formation as shown by the method described in FIG. 1 (data not shown). As the stability data indicates in FIG. 17, unformulated curcumin degrades very rapidly in aqueous buffers with ˜30% and 10% of initial curcumin present within 2 h and 24 h of incubation respectively. In contrast, the degradation of curcumin was significantly reduced when complexed with Eudragit® S100 as shown by the presence of ˜85-90% curcumin after 24 h of incubation, both in SIF and pH 7.0 buffer. Similar results were observed for Ora-Curcumin-E (FIG. 8)

### Physicochemical Characterization of CEMCs

Fourier Transform Infrared (FTIR) Spectroscopy

To ascertain the formation of CEMCs, FTIR spectrum of curcumin, Eudragit® S100 polymer, the physical mixture of curcumin and Eudragit® S100, and curcumin-polymer (Eudragit® 5100) molecular complexes were performed using Nicolet 380 ATR-FTIR spectrophotometer (Thermo Electron Corp., Madison, Wis.). Data was acquired between 4000 cm-1 and 400 cm-1 at a scanning speed of 4 cm-1 and 50 scans. The average of 50 scans data was presented.

FTIR spectroscopy was performed to ascertain the formation of complexes and the interaction pattern driving the complex formation. Curcumin (second from top)), Eudragit® S100 polymer (top)), the physical mixture of curcumin and Eudragit® S100 (third from top) and CEMCs (bottom)), were recorded for FTIR (FIG. 12). The phenolic —OH band characteristic of curcumin was diminished in the molecular complexes, which may be due to the formation of intermolecular hydrogen bonds between the phenolic —OH group of curcumin and the —C═O group of Eudragit® S100 backbone.

Nuclear Magnetic Resonance (NMR) Spectroscopy

To estimate the nature of interaction driving the formation of molecular complexes solution 1H NMR spectra of curcumin-polymer (Eudragit® S100) molecular complexes were recorded on a Bruker 400 MHz NMR spectrometer. Briefly, 30 mg of curcumin-polymer (Eudragit® S100) molecular complexes were dissolved in D2O: 0.5N Na2CO3 (1:1) followed by addition of DMSO-d6 to observe the decomplexation of the molecular complexes. Sample solution was transferred to a NMR tube and the spectrum was recorded.

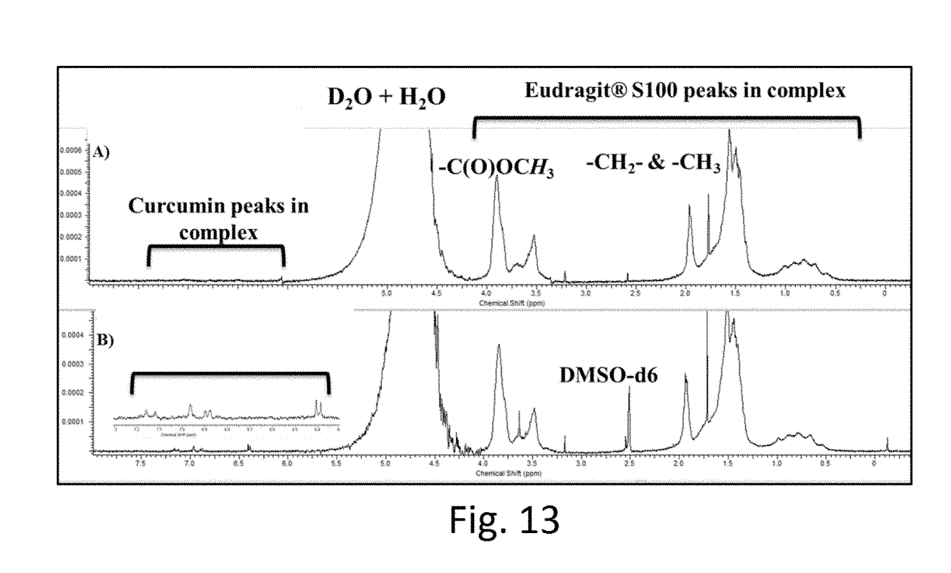

Solution 1H NMR spectra of curcumin-polymer (Eudragit® S100) molecular complexes was performed to ascertain the interaction pattern driving the molecular complex formation. The interaction pattern between curcumin and Eudragit® S100 polymer using 1H NMR. Molecular complexes were taken in 1 ml of D2O: 0.5N Na2CO3 (1:1). To this solution various amount of DMSO-d6 was added to disrupt the interaction. The signal that represents the aromatic region (6.0-7.5 ppm) was broad in D2O: 0.5N Na2CO3 (1:1) and starts appearing sharp as DMSO-d6 was added to the same sample (FIG. 13). This indicates that the complexation involves strong hydrophobic interactions between aryl skeleton of curcumin and alkyl chain of Eudragit® 5100. With the addition of organic solvent like DMSO, the complex might have broken down into curcumin and the polymer that might have made curcumin peaks to appear sharp. The hydrogen bonding interaction between hydroxyl group of curcumin and Eudragit® S100 is merely not the reason of complexation.

Scanning Electron Microscopy (SEM)

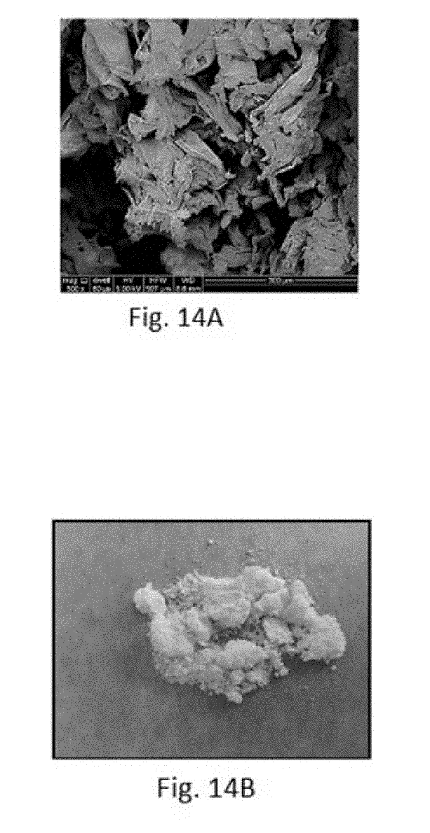

The surface morphology CEMCs was investigated using a scanning electron microscopy at an accelerating voltage of 5-10 kV, working distance of 5-15 mm and spot size of 3 (FIG. 14). The dry samples were mounted on metal holders using conductive double-sided tape and sputter coated with a gold layer for analysis.

Differential Scanning Calorimetric (DSC) Analysis

The physico-chemical nature of the molecular complexes was assessed using differential scanning calorimetry (DSC) analysis. The DSC analysis of curcumin, Eudragit® S100 polymer, the physical mixture of curcumin and Eudragit® S100, and CEMCs were performed using TA Instruments Q200 Differential Scanning calorimeter (TA Instruments, New Castle, Del., USA). Samples were weighed (equivalent to curcumin) and placed in sealed Tzero aluminum hermetic pans. With liquid nitrogen as coolant, samples were scanned at 10° C./min from −20° C. to 300° C. and thermograms were recorded.

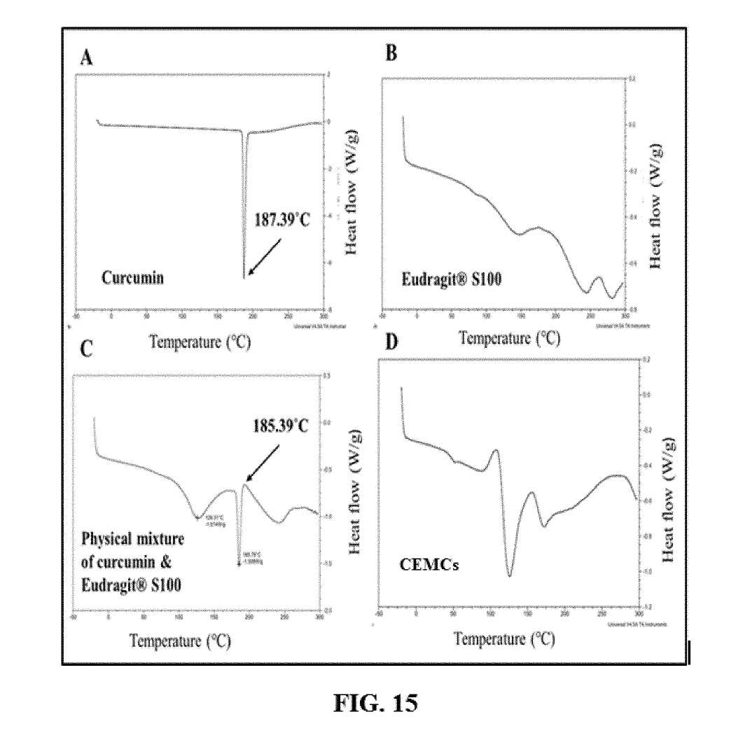

In FIG. 15, the DSC curves of curcumin (DSC curve A), Eudragit® 5100 polymer (DSC curve B), the physical mixture of curcumin and Eudragit® 5100 (DSC curve C), and curcumin-polymer (Eudragit® S100) molecular complexes according to the present invention (DSC curve D) were recorded. Curcumin (183° C.) (DSC curve A) showed a sharp melting peak at 187.39° C., indicating its crystalline nature. Eudragit® 5100 polymer (DSC curve B) did not exhibit a distinct melting point indicating the amorphous nature of the polymer. The physical mixture of curcumin and Eudragit® 5100 showed (DSC curve C) a sharp melting peak of curcumin at 185.39° C. and the relaxation peak of Eudragit® 5100. This indicates that in physical mixture curcumin is in crystalline form and Eudragit® S100 in amorphous form. However, such distinct melting point peak of crystalline curcumin was absent in curcumin-polymer (Eudragit® S100) (DSC curve D) suggesting a complete amorphous nature of both curcumin and Eudragit® S100 polymer in the complex form.

Powder X-Ray Diffraction (XRD)

Powder X-ray diffraction measurements of curcumin, Eudragit® S100 polymer, the physical mixture of curcumin and Eudragit® S100, and CEMCs were recorded using Rigaku powder x-ray diffractometer using Cu radiation, running at 40 kV and 44 mA. Samples were mounted on double sided silicone tape and measurements were performed from 2° C. to 60° C. at a scan speed of 4° C./min and increments of 0.02° C.

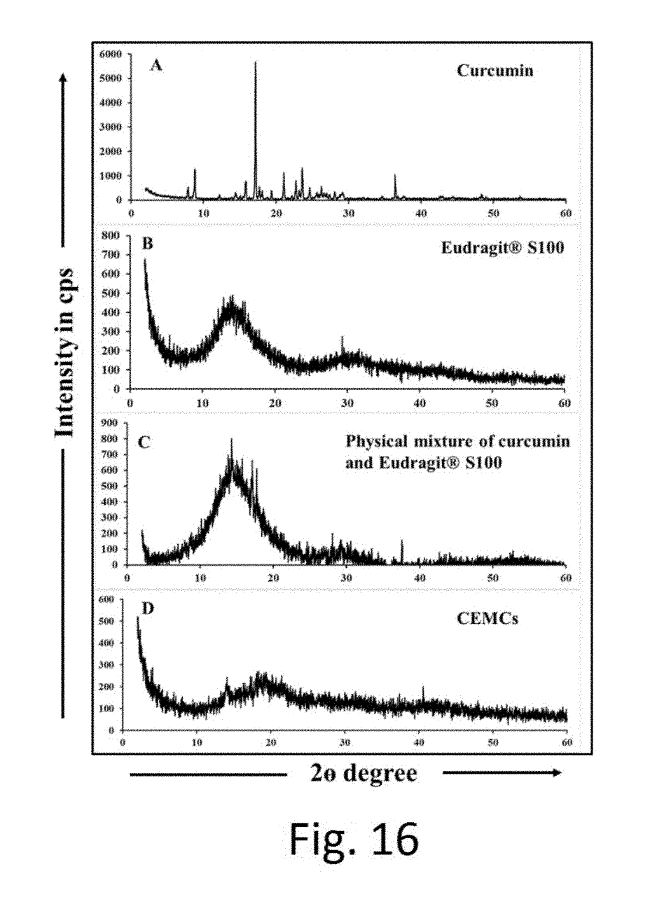

In FIG. 16, the powder X-ray diffraction patterns of curcumin showed characteristic sharp peaks indicative of crystalline nature of curcumin (pattern A). Eudragit® S100 did not show distinct peaks in XRD because of it is existence in amorphous form (pattern B). The peaks representing the crystalline nature of curcumin are visible in the physical mixture of synthetic curcumin and Eudragit® S100 (pattern C). In contrast, in molecular complexes of curcumin-polymer (Eudragit® S100) did not show any distinct peaks, which, further confirms the existence of curcumin and the polymer in amorphous form in molecular complexes (pattern D).

### Example 9

### pH Dependent Intracellular Delivery of Curcumin by Ora-Curcumin-S

The pH dependent cellular uptake of curcumin and Ora-Curcumin-S were determined on HCT116 and HT29 colo-rectal cancer cells by using flow cytometry (BD Biosciences FACS LSR Fortessa SORP Analyzer) as summarized in Table 6. Briefly, 2×10⁵ cells were seeded in 24 well-plates and allowed to adhere for 24 h. The cells were incubated with following groups: curcumin in pH 5.5 and pH 7.0 buffer solution and curcumin equivalent of Ora-Curcumin-S in pH 5.5 and pH 7.0 buffer solutions respectively at 37° C. After 4 h, the cells were washed with PBS, trypsinized, fixed using 4% paraformaldehyde (PFA) and stored at 4° C. until further analysis using flow cytometer. The amount of curcumin or Ora-Curcumin-S received per cell was quantified using flow cytometer equipped with a blue laser (488 nm). Sample acquisition was performed using BD Biosciences FACS LSR Fortessa and data analyzed using FlowJo software (FlowJo LLC).

Ora-Curcumin-S represents highly soluble and stable form of curcumin; it was expected to enhance the intracellular delivery of curcumin. The uptake of Ora-Curcumin-S into the human colo-rectal cancer cells, HT29 and HCT116 was compared with unformulated curcumin at pHs 5.5 and 7.0. The pH 5.5 and 7.0 represents the luminal pHs of the proximal and distal intestine. After 4 h of incubation, Ora-curcumin-S was highly efficient in delivering the curcumin to the colo-rectal cancer cells as shown in Table 6. Ora-curcumin-S on average delivered ˜10 times (HT29 colo-rectal cancer cells) and ˜5 times more curcumin to each cell compared to unformulated curcumin when delivered through pH 5.5 and pH 7.0 solution, respectively.

TABLE 6
Cellular uptake of curcumin on HCT116 and HT29 colo-rectal cancer cells

| | Mean fluorescence intensity | | | |
|---|---|---|---|---|
| | HT29 cells | | HCT116 cells | |
| Groups | pH 5.5 | pH 7.0 | pH 5.5 | pH 7.0 |
| Unformulated | 2704 ± 221 | 2429 ± 247 | 2543 ± 72 | 3514 ± 975 |
| Curcumin | | | | |
| Ora-Curcumin-S | 7188 ± 1398*** | 20998 ± 2210*** | 3555 ± 1168 | 8783.67 ± 1760*** |

Data represents mean ± standard deviation (n = 3).
***indicates that the values are significantly higher compared to all other groups (p ≤ 0.001) calculated using one-way ANOVA followed by Bonferroni's post-hoc multiple comparison test.

### Example 10

### Ora-Curcumin is Biologically Active as a Toll-Like Receptor-4 (TLR-4) Antagonist.

Accumulating evidence from the published literature strongly suggests the role of TLR-4 in the etiology of IBD, colo-rectal cancer and other inflammatory diseases. In addition, dendritic cells play a significant role in the initiation of inflammation in IBD. The DC2.4 cells represent naïve dendritic cells from mouse origin. DC2.4 cells and HEKTLR-4^{YFP}MD2 cells were used to assess the TLR-4 antagonistic activity of Ora-Curcumin-S. HEKTLR-4^{YFP}MD2 cells are genetically modified human kidney epithelial cells that exclusively express only TLR-4 receptors on their surface among all the TLRs and are capable of downstream signaling upon the activation of TLR on its surface.

A total of 5.0×10⁵ mouse naïve dendritic cells (DC2.4) were treated with; a) Medium alone b) curcumin (5 and 10 μg/ml) c) curcumin equivalent of Ora-Curcumin-S (5 and 10 μg/ml). Post 1 h of incubation, cells were treated with MPLA (2 μg/ml) or (5.0×10⁵ E. coli/ml). After 48 h, the concentration of TNF-α in the culture supernatant was measured using TNF-α ELISA Ready-SET-Go kit.

The dose-response curve of curcumin or Ora-Curcumin-S on inhibiting the MPLA induced activation of genetically modified HEK293-TLR4^{YFP}MD2 cells was assessed by treating 5.0×10⁵ cells/well with either curcumin or curcumin equivalent of Ora-Curcumin-S at concentrations of 1, 2, 5, 10, 30 and 50 μg/ml. Post 1 h of incubation, cells were treated with MPLA (2 μg/ml) to activate the cells. After 48 h, the concentration of IL-8 in the culture supernatant was measured using IL-8 ELISA Ready-SET-Go kit as an indicator of TLR-4 activation.

In another separate set of experiments, we determined the effect of varying concentrations of MPLA and constant amounts of curcumin or curcumin equivalent of Ora-Curcumin-S on IL-8 release. A total of 5.0×10⁵ HEK293TLR4YFPMD2 cells were treated with 5 μg/ml of curcumin or curcumin equivalent of Ora-Curcumin-S. After 1 h, the cells were treated with varying concentrations of MPLA (3, 4, 5 and 6 μg/ml) for 48 h. At the end, the concentration of IL-8 in the culture supernatant was measured using ELISA as an indicator of TLR-4 activation.

In the dose-response study using constant amounts of MPLA (2 μg/ml) in HEK293-TLR4^{YFP}MD2 cells, Ora-Curcumin-S significantly enhanced the activity of curcumin at equivalent curcumin concentrations of 5 μg/ml and above as shown in FIG. 18. Ora-Curcumin-S was approximately 2 to 2.5 times more potent at concentrations of 5, 10, 30 and 50 μg/ml in inhibiting the TLR-4 receptors as compared to curcumin alone when incubated in presence of constant amounts of MPLA 2 μg/ml.

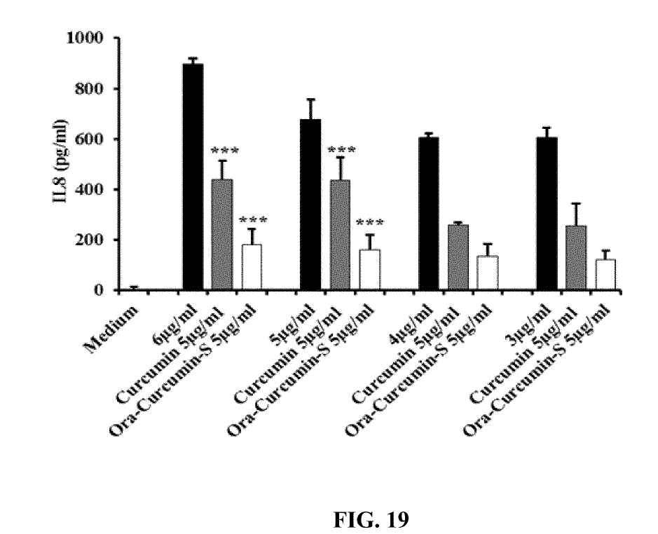

The data for the other set of samples with cells exposed to a constant amount of curcumin or curcumin equivalent of Ora-Curcumin-S (5 μg/ml) and challenged with increased concentrations of MPLA (3-6 μg/ml) is shown in FIG. 19.

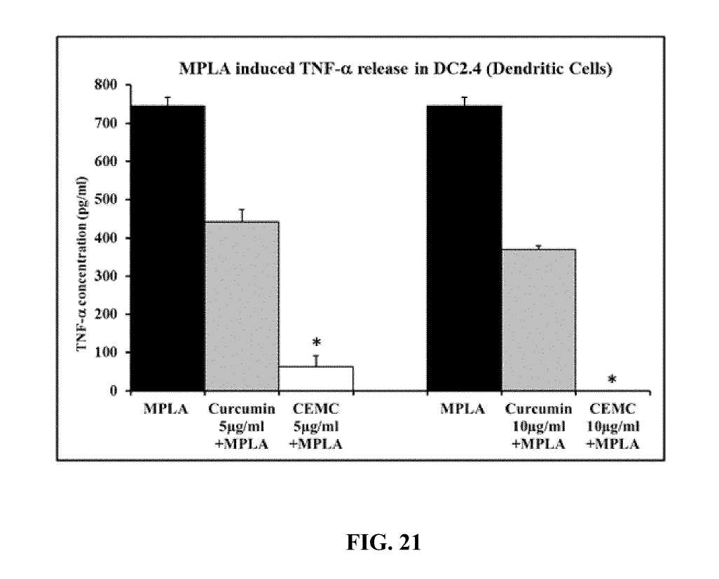

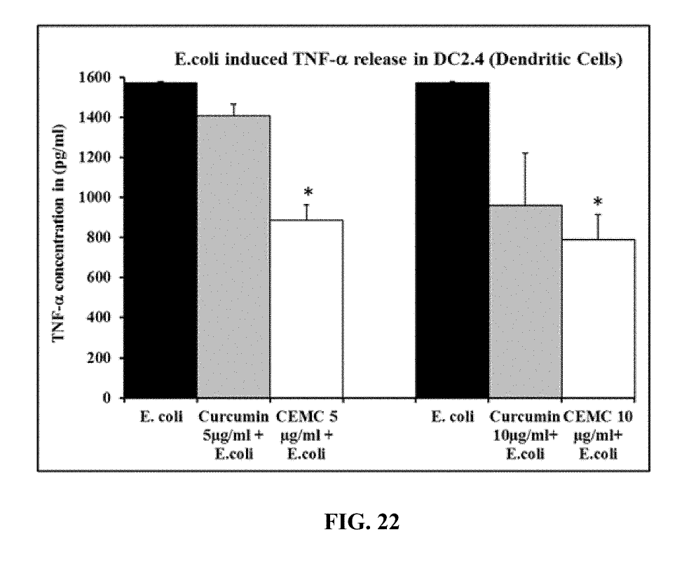

The TLR-4 modulating activity of Ora-Curcumin-S and its benefit as an anti-inflammatory formulation was assessed by its ability in inhibiting the TLR-4-mediated inflammatory response in dendritic cells. Activated dendritic cells secret pro-inflammatory cytokines such as TNF-alpha. MPLA and E. Coli stimulated the DC2.4 cells to release high amounts of TNF-alpha through the activation of TLR-4. As shown in FIG. 21, Ora-Curcumin-S (5 μg/ml) was ˜10-15 times more potent in inhibiting the TNF-α release as compared to soluble curcumin alone in response to MPLA challenge (2 μg/ml). Similar to inhibiting the MPLA induced inflammation, as shown in FIG. 22, Ora-Curcumin-S significantly inhibited the activation of dendritic cells (TNF-α release) when exposed to dead E. coli that is also known to activate TLR-4. Ora-Curcumin-S was ˜2.5 times more potent than soluble curcumin in antagonizing dendritic cell activation in response to E. Coli exposure.

It was found that CEMCs inhibits TLR-4 stimulation in genetically modified TLR-4 cells by TLR-4 agonists (MPLA).

### Example 11

### Ora-Curcumin-S Enhanced the Anticancer Activity of the Curcumin

The effect of curcumin and Ora-Curcumin-S on the viability of HCT116 and HT29 human colo-rectal cancer cells was determined by the 3-(4, 5-dimethylthiazol-2-yl)-2, 5-diphenyltetrazolium bromide (MTT) assay method. Curcumin was dissolved either in dimethyl sulfoxide (DMSO) or pH 7.0 buffer, and Ora-Curcumin-S was dissolved in pH 7.0 buffer as a stock solution. Briefly, 50,000 cells/well were incubated with varying concentrations of curcumin and curcumin equivalent of Ora-Curcumin-S at 37° C. Following 48 h of incubation, 0.5 mg/ml MTT solution was added to each well, and the cells were incubated for further 4 h at 37° C. After 4 h, 150 μl of DMSO was added to each well and incubated for 1 h at 37° C. to dissolve the formazan crystals. The absorbance was then measured at 570 nm with a reference wavelength of 650 nm using UV-visible spectrophotometer. The results are represented in terms of percentage inhibition of cell proliferation compared to that of vehicle control. The cellular viability was assessed as a percentage of the control by using the following equation.

$$Viability ( \% ) = \frac{\begin{matrix}Absorbance of cells treated with curcumin or \\ curcumin \text{-} Eudragit ® S 100 complexes\end{matrix}}{Absorbance of the untreated cells} \times 100$$

In this experiment, Ora-Curcumin-S was dissolved in pH 7.0 buffer. Since unformulated curcumin is not soluble in pH 7.0 buffer, two different stocks were prepared by solubilizing the curcumin in DMSO and dispersing in pH 7.0 buffer. As expected, dispersed curcumin in pH 7.0 buffer did not suppress the proliferation of HT29 and HCT116 cells, as there was very little soluble curcumin to enter the cells. However, curcumin dissolved in DMSO and curcumin equivalent Ora-Curcumin-S in pH7.0 solution suppressed the proliferation of HT29 and HCT116 cells in a dose-dependent manner, as shown in Table 7.

TABLE 7
Anti-cancer Activity.

| | IC₅₀ (mean ± SD) in μM | IC₅₀ (mean ± SD) in μM |
|---|---|---|
| Groups | In HT29 Cells | In HCT116 Cells |
| Unformulated | NA* | NA* |
| Curcumin in pH 7.0 | | |
| buffer | | |
| Curcumin in DMSO | 41.84 ± 1.86 | 18.35 ± 1.94 |
| Ora-Curcumin-S in | 17.47 ± 0.63 | 10.39 ± 1.08 |
| pH 7.0 buffer | | |

Compared to curcumin in DMSO (IC₅₀ for HT29 41.84±1.86 μM and HCT116 18.35±1.94 μM), Ora-Curcumin-S (IC₅₀ for HT29 17.47±0.63 μM and HCT116 10.39±1.08 μM) showed a significant reduction in cell viability of colo-rectal cancer cells. The stronger inhibitory effect of Ora-Curcumin-S could be attributed to the enhanced cellular uptake of curcumin through Ora-Curcumin-S delivery, which could be because of improved aqueous solubility and stability Ora-Curcumin-S as compared to unformulated curcumin. The concentration of curcumin that reduced the viability of cells by 50% of initial viability (IC₅₀) for unformulated curcumin (in pH7.0 buffer or DMSO) or curcumin equivalent of Ora-Curcumin-S (pH 7.0 buffer) on HT29 and HCT116 human colo-rectal cancer cells. Data represents mean±standard deviation (n=4-5). Unformulated curcumin at pH 7.0 did not altered the viability below 50% at all concentrations tested (0-80 μM).

### Example 12

### In-Vivo Plasma Concentration of Curcumin and Curcumin Equivalent of Ora-Curcumin-S in Mice

BALB/cJ mice (n=5 per group, 6-8 weeks old) purchased from Jackson laboratories (Maine, USA) were used for the estimation of the plasma concentration. Curcumin (15 mg/kg) or equivalent Ora-Curcumin-S was orally administered. Blood samples from mice were collected by tail snip method at 1 h after administering the formulations. The curcumin present in the mouse plasma was immediately extracted with acetonitrile and the amount of curcumin was determined by reverse phase high-performance liquid chromatography (HPLC) method as explained below. All animal experimentation was performed in compliance with regulations of the Institutional Animal Care and Use Committees (IACUC) of South Dakota State University, Brookings, S. Dak., USA.

Quantification of curcumin was performed by reverse phase HPLC using chromatographic separation on a Symmetry® C18 column (150 mm×4.6 mm, 5 μm, Waters, USA) with an isocratic elution using mobile phase composed of acetonitrile and 1% w/v citric acid buffer (pH 3.0) (60:40 v/v ratio). The flow rate was set at 1 ml/min. Curcumin was detected using a UV detector at 420 nm with a sample volume of 50 μl per injection.

As shown in FIG. 23, Ora-Curcumin-E enhanced the blood levels of curcumin after 1 hour of administration as expected, whereas Ora-Curcumin-S did not show any detectable levels of plasma curcumin. However, Ora-Curcumin-S is targeted to the lumen of the colon where the inflammation in IBD and colo-rectal cancer is located, instead of exposing the whole body through blood circulation as by Ora-Curcumin-E (curcumin complex with Eudragit® EPO).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. §112(f) are not to be invoked unless the specific terms “means for” or “step for” are recited in a claim.

1. A method of inhibiting the stimulation of TLR receptors in a patient, the method comprising:
providing a curcuminoid-polymer complex in a medicament, the curcuminoid-polymer 5 complex comprising at least one curcuminoid component selected from the group consisting essentially of curcumin, demethoxycurcumin, bisdemethoxycurcumin, tetrahydroxycurcumin, Bis-0-Demethyl curcumin (BDMC), or combinations thereof, at least one polymer or co-polymer component having a backbone comprising polymethacrylate or methyl methacrylate, and optionally a surfactant component; and
delivering the curcuminoid-polymer complex to a gastro-intestinal tract of said patient.

2. The method of claim 1, wherein the curcuminoid component comprises curcumin.

3. The method of claim 2, wherein the complex presents an aqueous solubility of the curcumin in an amount greater than about 1 μg/ml.

4. The method of claim 3, wherein the aqueous solubility is between about 1 μg/ml and about 100 mg/ml.

5. The method of claim 1, wherein the polymer or co-polymer component is chosen such that the aqueous solubility of the curcuminoid component is greater than about 1 μg/ml at a pH of 7.0 and above.

6. The method of claim 1, wherein the polymer or co-polymer component is chosen such that the aqueous solubility of the curcuminoid component is greater than about 1 μg/ml at a pH between about 1.0 and about 14.0.

7. The method of claim 1, wherein the polymer or co-polymer comprises a cationic or anionic copolymer based on methacrylic acid and methyl methacrylate.

8. The method of claim 1, comprising a first polymer or co-polymer component and a second polymer or co-polymer component, the first polymer or co-polymer forming a first complex and the second polymer or co-polymer forming a second complex, wherein the first polymer or co-polymer component is chosen such that the aqueous solubility of the curcuminoid component of the first complex is greater than about 1 μg/ml at a pH between about 1.0 and about 5.0, and wherein the second polymer or co-polymer component is chosen such that the aqueous solubility of the curcuminoid component of the second complex is greater than about 1 μg/ml at a pH between about 5.5 and about 14.0.

9. The method of claim 1, wherein a weight ratio of the curcuminoid component to the polymer or co-polymer component used in a preparation of the curcuminoid-polymer complex is between about 1:0.1 to about 1:50.

10. The method of claim 1, wherein the complex increases the stability of the curcuminoid component in an aqueous solution compared to free curcumin.

11. The method of claim 1, further comprising at least one adjuvant.

12. The method of claim 1, wherein the medicament is in the form of a solid, a liquid, or a semisolid.

13. The method of claim 1, wherein the curcumin of the curcuminoid-polymer complex delivered to the gastro-intestinal tract inhibits the activation of TLR receptors.

14. The method of claim 1, wherein the curcumin of the curcuminoid-polymer complex delivered to the gastro-intestinal tract antagonizes TLR4 activation.

15. The method of claim 1, wherein the curcumin of the curcuminoid-polymer complex delivered to the gastro-intestinal tract reduces the release of pro-inflammatory cytokines by immune cells.

16. A method of treating a disease or disorder mediated by inflammatory cytokines in a patient, the method comprising:
administering to a gastro-intestinal tract of said patient a medicament having a plurality of curcuminoid-polymer complex particles, the curcuminoid-polymer complex comprising at least one curcuminoid component selected from the group consisting essentially of curcumin, demethoxycurcumin, bisdemethoxycurcumin, tetrahydroxycurcumin, Bis-0-Demethyl curcumin (BDMC), or combinations thereof, at least one polymer or co-polymer component having a backbone comprising polymethacrylate or methyl methacrylate, and a surfactant component, wherein an aqueous solubility of the curcuminoid component of the curcuminoid-polymer complex is greater than about 1 μg/ml at a pH between about 2.0 and about 14.0.

17. The method of claim 16, wherein the curcuminoid component comprises curcumin.

18. The method of claim 17, wherein the polymer or co-polymer comprises an copolymer based on methacrylic acid and methyl methacrylate.

19. The method of claim 18, wherein an aqueous solubility of curcumin is greater than about 1 μg/ml at a pH between about 5.0 and about 14.0.

20. The method of claim 16, wherein the disease or disorder is selected from mucositis, sepsis, gastritis, irritable bowel syndrome, inflammatory bowel disease, bacterial infections, and cancers of the gastro-intestinal tract.
